(19) United States
(12) Patent Application Publication
(10) Pub. No.: US 2025/0043337 A1
(43) Pub. Date: Feb. 6, 2025

(54) METHODS OF ANALYSIS OF METHYLATED DNA BINDING PROTEINS

(71) Applicant: GUARDANT HEALTH, INC., Palo Alto, CA (US)

(72) Inventors: Saiyou OHSHIMA, Morgan Hill, CA (US); Kenneth Edmund STAPLETON, San Francisco, CA (US); Dustin Howard HITE, Redwood City, CA (US); Xiao-Bo CHEN, Foster City, CA (US); Chia-Hui LIN, San Mateo, CA (US); Dania ANNUAR, Oakland, CA (US); Jessica Michelle PIERACCI, Salinas, CA (US)

(21) Appl. No.: 18/661,147

(22) Filed: May 10, 2024

Related U.S. Application Data
(63) Continuation of application No. PCT/US2022/079757, filed on Nov. 11, 2022.
(60) Provisional application No. 63/279,001, filed on Nov. 12, 2021.

(51) Int. Cl. C12Q 1/6834; C12Q 1/6806; G01N 21/64; G01N 33/58

(52) U.S. Cl. CPC C12Q 1/6834; C12Q 1/6806; G01N 21/6486; G01N 33/58; C12Q 2600/154

(57) ABSTRACT

Provided herein are methods of assaying a methylated DNA-binding protein comprising contacting methylated DNA-binding proteins with labeled oligonucleotides, obtaining sample partitions for oligonucleotides with different methylation states, and quantifying the labeled oligonucleotides in the partitions.

### CROSS-REFERENCE TO RELATED APPLICATIONS

This application is a continuation of International PCT Application No. PCT/US2022/079757, filed Nov. 11, 2022, which claims the benefit of U.S. Provisional Patent Application No. 63/279,001, filed Nov. 12, 2021, which is incorporated herein by reference for all purposes.

### FIELD OF THE INVENTION

This disclosure relates to methods for assaying methylated DNA binding proteins.

### INTRODUCTION AND SUMMARY

Cancer is responsible for millions of deaths per year worldwide. Early detection of cancer may result in improved outcomes because early-stage cancer tends to be more susceptible to treatment.

Improperly controlled cell growth is a hallmark of cancer that generally results from an accumulation of genetic and epigenetic changes. In particular, cancer can be indicated by non-sequence modifications, such as changes to DNA methylation. Examples of methylation changes in cancer include local gains of DNA methylation in the CpG islands at the transcription start site (TSS) of genes involved in normal growth control, DNA repair, cell cycle regulation, and/or cell differentiation. This hypermethylation can be associated with an aberrant loss of transcriptional capacity of involved genes and occurs at least as frequently as point mutations and deletions as a cause of altered expression.

DNA methylation profiling can be used to detect aberrant methylation in DNA of a sample. A variety of methods are available for analyzing methylated DNA, including methylated-DNA enrichment methods that employ methylated DNA binding proteins (MBPs), such as methylated-CpG binding proteins. However, MBPs can show lot-to-lot variability, e.g., in terms of their binding activity toward methylated DNA. Accordingly, there is a need for improved methods for assessing the binding efficiency of MBPs, including methods that relate to determining a quantitative unit of binding activity for MBPs. The present disclosure aims to meet this need, provide other benefits, or at least provide the public with a useful choice. Accordingly, the following exemplary embodiments are provided.

Embodiment 1 is a method of assaying a methylated DNA-binding protein, comprising: (a) contacting the methylated DNA-binding protein with a sample comprising at least a first oligonucleotide; (b) obtaining at least hypomethylated and hypermethylated partitions of the sample; and (c) quantifying the first oligonucleotide in the hypomethylated and hypermethylated partitions.

Embodiment 2 is the method of embodiment 1, wherein the methylated DNA-binding protein is immobilized on a solid support.

Embodiment 3 is the method of embodiment 2, wherein the solid support comprises plate wells.

Embodiment 4 is the method of any one of embodiments 1-3, wherein the first oligonucleotide is quantified by measuring absorbance.

Embodiment 5 is the method of any one of embodiments 1-4, wherein the first oligonucleotide is labeled.

Embodiment 6 is the method of embodiment 5, wherein the first oligonucleotide is fluorescently or radioactively labeled.

Embodiment 7 is the method of any one of embodiments 1-6, wherein (a) the hypomethylated partition comprises a first salt concentration, and (b) the hypermethylated partition comprises a second salt concentration, wherein the second salt concentration is higher than the first salt concentration.

Embodiment 8 is the method of embodiment 7, wherein (a) the first salt concentration ranges from 0 M to 1 M and/or (b) the second salt concentration ranges from 1.0 M to 3.0 M.

Embodiment 9 is the method of embodiment 5, wherein (a) the first salt concentration ranges from 0 M to 0.6 M and/or (b) the second salt concentration ranges from 0.6 M to 3.0 M.

Embodiment 10 is the method of any one of embodiments 5-7, wherein the first salt concentration is about 0.3 M.

Embodiment 11 is the method of any one of embodiments 5-8, wherein the second salt concentration is about 2 M.

Embodiment 12 is the method of any one of embodiments 5-9, wherein the salt is a sodium salt.

Embodiment 13 is the method of embodiment 12, wherein the salt is NaCl.

Embodiment 14 is the method of any one of embodiment 1-13, wherein the first oligonucleotide is methylated.

Embodiment 15 is the method of embodiment 14, wherein the sample further comprises a labeled unmethylated oligonucleotide and the first methylated nucleotide is labeled, further wherein the labeled unmethylated oligonucleotide is differentially labeled relative to the first methylated oligonucleotide.

Embodiment 16 is the method of embodiment 15, further comprising quantifying the labeled unmethylated oligonucleotide in the hypomethylated and hypermethylated partitions.

Embodiment 17 is the method of any one of embodiments 1-16, wherein the first methylated oligonucleotide comprises a methylated CpG.

Embodiment 18 is the method of any one of embodiments 1-17, wherein the first methylated oligonucleotide comprises at least 2 methylated CpGs.

Embodiment 19 is the method of any one of embodiments 1-18, wherein the first methylated oligonucleotide comprises 2, 3, 4, 5, 6, 7, 8, 9, 10, 11, or 12 methylated CpGs.

Embodiment 20 is the method of any one of embodiments 1-19, wherein the first methylated oligonucleotide comprises 3 or 9 methylated CpGs.

Embodiment 21 is the method of any one of embodiments 14-20, wherein the first methylated oligonucleotide is labeled and the sample further comprises a second labeled methylated oligonucleotide, wherein the second labeled methylated oligonucleotide has a greater number of methylated positions than the first labeled methylated oligonucleotide, further wherein the second labeled methylated oligonucleotide is differentially labeled relative to the first labeled methylated oligonucleotide.

Embodiment 22 is the method of embodiment 21, further comprising quantifying the second labeled methylated oligonucleotide in the hypomethylated and hypermethylated partitions.

Embodiment 23 is the method of embodiment 19 or 20, wherein the sample further comprises a labeled unmethylated oligonucleotide, wherein the labeled unmethylated oligonucleotide is differentially labeled relative to the first labeled methylated oligonucleotide and the second labeled methylated oligonucleotide.

Embodiment 24 is the method of embodiment 23, further comprising quantifying the labeled unmethylated oligonucleotide in the hypomethylated and hypermethylated partitions.

Embodiment 25 is the method of any one of embodiments 19-24, wherein each of the first and second labeled methylated oligonucleotides comprises at least one methylated CpG.

Embodiment 26 is the method of any one of embodiments 19-25, wherein each of the first and second labeled methylated oligonucleotides comprises at least 2 methylated CpGs.

Embodiment 27 is the method of any one of embodiments 19-26, wherein each of the first and second labeled methylated oligonucleotide comprises 2, 3, 4, 5, 6, 7, 8, 9, 10, 11, or 12 methylated CpGs.

Embodiment 28 is the method of any one of embodiments 19-27, wherein the first labeled methylated oligonucleotide comprises 3 methylated CpGs.

Embodiment 29 is the method of any one of embodiments 19-27, wherein the second labeled methylated oligonucleotide comprises 9 methylated CpGs.

Embodiment 30 is the method of any one of embodiments 1-29, wherein the first and/or second labeled oligonucleotide has a length of at least 10, at least 11, at least 12, at least 13, at least 14, at least 15, at least 16, at least 17, at least 18, at least 19, at least 20, at least 30, at least 40, at least 50, at least 70, at least 100, or at least 130 nucleotides.

Embodiment 31 is the method of any one of embodiments 1-30, further comprising obtaining an intermediate partition.

Embodiment 32 is the method of any one of embodiments 1-31, wherein the first methylated oligonucleotide and/or the second labeled methylated oligonucleotide binds to a methyl binding domain, a methyl binding protein, and/or an antibody that preferentially binds to 5-methylcytosine (5mC) over unmodified cytosine.

Embodiment 33 is the method of any one of embodiments 1-32, wherein the methylated DNA binding protein comprises MeCP2, MBD1, MBD2, MBD4, or a methyl-CpG binding zinc finger protein. Embodiment 33.1 is the method of any one of embodiments 1-32, wherein the methylated DNA binding protein comprises an antibody that binds to methylated cytosine.

Embodiment 34 is the method of any one of embodiments 1-33.1, further comprising measuring a binding activity of the methylated DNA binding protein.

Embodiment 35 is the method of embodiment 34, wherein the binding activity is determined in units indicating an amount of protein capable of binding a predetermined fraction of a reference methylated oligonucleotide.

Embodiment 36 is the method of embodiment 35, wherein the predetermined amount ranges from at least 5% to 95% or at least 10% to 90% of the reference methylated oligonucleotide.

Embodiment 37 is the method of embodiment 38, wherein the predetermined amount is about 50%, 60%, 70%, 80%, or 90% of the reference methylated oligonucleotide.

Embodiment 38 is the method of any one of embodiments 35-37, wherein the reference methylated oligonucleotide comprises at least 1 or at least 2 methylated CpGs.

Embodiment 39 is the method of any one of embodiments 35-38, wherein the reference methylated oligonucleotide comprises 1, 2, 3, 4, 5, 6, 7, 8, 9, 10, 11, or 12 methylated CpGs.

Embodiment 40 is the method of any one of embodiments 35-39, wherein the reference methylated oligonucleotide comprises 3 or 9 methylated CpGs.

Embodiment 41 is the method of any one of embodiments 35-40, wherein the reference methylated oligonucleotide has a length of at least 10, at least 11, at least 12, at least 13, at least 14, at least 15, at least 16, at least 17, at least 18, at least 19, or at least 20 nucleotides on each strand.

Embodiment 42 is the method of any one of embodiments 1-41, wherein the first oligonucleotide, the second labeled methylated oligonucleotide, the unmethylated oligonucleotide, and/or the reference methylated oligonucleotide is double-stranded.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings, which are incorporated in and constitute a part of this specification, illustrate one (several) embodiment(s) and together with the description, serve to explain the principles described herein.

### BRIEF DESCRIPTION OF THE DRAWINGS

FIG. 1 shows an exemplary workflow for a method of assaying a methylated DNA-binding protein according to some embodiments of the disclosure. MW1=0.3 M NaCl solution; hypomethylated partition. MW3=2 M NaCl; hypermethylated partition. P=biotinylated methylated DNA binding proteins (MBPs). Black-filled circles=unbound DNA probes. Checkered circles=DNA probes released into MW3. In some embodiments, the amount of MBP is varied, e.g., over a 2-fold dilution series.

FIGS. 2A-C show exemplary workflows according to certain embodiments of the disclosure. FIGS. 2A-B show embodiments using biotin-labeled MBPs captured by streptavidin beads (MBD-beads) and one or more DNA probes. FIG. 2A shows an embodiment using a single DNA probe comprising three methylated CpGs and a FAM fluorophore (N3 FAM). FIG. 2B shows an embodiment using a triplex DNA probe mix comprising NO-FAM, N3-Cy5, and N9-HEX DNA probes (0, 3, and 9 CpGs, respectively; and FAM, Cy5, and HEX fluorophores, respectively). FIG. 2C illustrates the bead-based and bead-independent embodiments of assays according to the disclosure. MW1=a solution comprising 0.3 M NaCl (used to obtain hypomethylated partition). MW3=a solution comprising 2 M NaCl (used to obtain hypermethylated partition). RT=room temperature.

FIG. 3 shows a graph for determining the amount of recovered DNA probes using measured mean relative fluorescence units (RFUs).

FIG. 4 shows a graph for assessing the binding affinity of MBPs for DNA probes.

FIG. 5 shows MBD binding units for two lots of MBPs, Lot 133 and Lot 148.

FIGS. 6A-B show the fraction of N3 probe recovered from MBD beads as a function of μL beads/ng N3 DNA. The confidence intervals at 50% of N3 DNA probes recovered for both Lot 133 and Lot 148 were at 95%. FIG. 6A: Lot 133. FIG. 6B: Lot 148.

FIG. 7 shows exemplary standard curves for concentrations of DNA probes in MW1 and MW3.

FIG. 8 shows exemplary standard curves for concentrations of DNA probes in MW1 and MW3.

FIGS. 9A-B show exemplary graphs for determining yield fraction (%) and RFU fraction (%).

FIG. 10 shows an exemplary graph of RFU across different partitions for a range of MBD-protein amounts.

FIG. 11 compares triple and single washes in the wash step of the MBD assay.

FIGS. 12A-B show the effect incubation times on DNA probe binding. FIG. 12A shows RFU for the fraction of bound and unbound DNA probes. FIG. 12B shows a magnified view of FIG. 12A, highlighting the shift in the 50% fraction point.

FIG. 13 shows the effect of increasing amounts of N3 FAM DNA probe on the 50% fraction point.

FIGS. 14A-B show the effect of incubation times on the DNA probes. FIG. 14A shows RFU for the fraction of bound and unbound DNA probes. FIG. 14B shows a magnified view of FIG. 14A to show data points around the 50% fraction point.

FIG. 15 shows exemplary binding curves and curve fitting using four-parameter (4P) and five-parameter (5P) algorithms. R² values are shown. '135 and '133 indicate distinct MBD-biotin lots.

FIGS. 16A-B show exemplary curves for titration with N3 DNA probes. FIG. 16A shows an exemplary curve for 50% yield fraction. FIG. 16B shows an exemplary curve for 90% yield fraction.

FIG. 17 shows exemplary binding curves and curve fitting using 4P and 5P algorithms. The amounts of N3 DNA probes used ranged from 13 ng to 78 ng and are shown for each curve.

FIG. 18 shows the ratios of MBD-biotin volumes of different lots at 50% yield fraction for two individual operators. 133, 135, 148, 679, and 855 indicate distinct MBD-biotin lots.

FIG. 19 shows an exemplary standard curve using 5P curve-fitting algorithm.

FIGS. 20A-C show results from an exemplary run of the bead-based MBD assay with a triple probe mix comprising NO-FAM, N3-Cy5, and N9-HEX. FIG. 20A shows hypomethylated partitions. FIG. 20B shows residual partitions. FIG. 20C shows hypermethylated partitions.

### DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

As used herein, “partitioning” of nucleic acids, such as DNA molecules, means separating, fractionating, sorting, or enriching a sample or population of nucleic acids into a plurality of subsamples or subpopulations of nucleic acids based on one or more modifications or features that is in different proportions in each of the plurality of subsamples or subpopulations. Partitioning may include physically partitioning nucleic acid molecules based on the presence or absence of one or more methylated nucleobases. A sample or population may be partitioned into one or more partitioned subsamples or subpopulations based on a characteristic that is indicative of a genetic or epigenetic change or a disease state.

As used herein, a “label” is a capture moiety, fluorophore, oligonucleotide, or other moiety that facilitates detection, separation, or isolation of that to which it is attached.

As used herein, a “capture moiety” is a molecule that allows affinity separation of molecules linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

The term “methylation” or “DNA methylation” refers to addition of a methyl group to a nucleobase in a nucleic acid molecule. In some embodiments, methylation refers to addition of a methyl group to a cytosine at a CpG site (cytosine-phosphate-guanine site (i.e., a cytosine followed by a guanine in a 5′->3′ direction of the nucleic acid sequence). In some embodiments, DNA methylation refers to addition of a methyl group to adenine, such as in N6-methyladenine. In some embodiments, DNA methylation is 5-methylation (modification of the 5th carbon of the 6-carbon ring of cytosine to create 5-methylcytosine (5mC)). In some embodiments, methylation comprises a derivative of 5mC. Derivatives of 5mC include, but are not limited to, 5-hydroxymethylcytosine (5-hmC), 5-formylcytosine (5-fC), and 5-caryboxylcytosine (5-caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

The term “hypermethylation” refers to an increased level or degree of methylation of DNA relative to the other DNA molecules within a population (e.g., sample) of DNA molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The terms “agent that recognizes a modified nucleobase in DNA,” such as an “agent that recognizes a modified cytosine in DNA” refers to a molecule or reagent that binds to or detects one or more modified nucleobases in DNA, such as methyl cytosine. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methyl cytosine. In such embodiments, the cytosine modification is a methyl. Agents that recognize a methyl cytosine in DNA include, but are not limited to, “methyl binding reagents,” which refer herein to reagents that bind to a methyl cytosine. Methyl binding reagents include, but are not limited to, methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded.

As used herein, “methylated DNA binding protein” refers herein to a polypeptide or combination thereof that binds to DNA comprising a methylated nucleotide, such as a methylcytosine. Methylated DNA binding proteins include, but are not limited to, methylated DNA binding domains (MBDs), methylated DNA binding proteins (MBPs), and antibodies specific for methylcytosine. In some embodiments, such MBDs, MBPs, and antibodies bind to 5-methylcytosine in DNA. In some such embodiments, the DNA may be single-stranded or double-stranded.

As used herein, “precision” refers to the ability of a measurement to be reproduced consistently. As used herein, “repeatability” refers to intra-assay precision. As used herein, “intermediate precision” refers to inter-assay precision.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### II. Exemplary Methods

Disclosed methods herein comprise analyzing methylated DNA-binding protein. In such methods, any methylated DNA-binding protein may be contacted with one or more oligonucleotides, e.g., oligonucleotides with different methylation states. In some embodiments, the methylated DNA-binding protein is immobilized on a solid support, such as a plate well or bead. In some embodiments, hypomethylated and hypermethylated partitions are obtained. In some embodiments, the one or more labeled oligonucleotides are quantified in the partitions. In some embodiments, the sample comprises oligonucleotides that comprise more than one methylation state, and the different oligonucleotides can be separated into partitions. In some embodiments, oligonucleotides are quantified by measuring absorbance. In some embodiments, oligonucleotides are labeled, e.g., fluorescently or radioactively. In such embodiments, the oligonucleotides may be quantified using a signal from the label.

### A. Oligonucleotides

Oligonucleotides may be single stranded or double stranded DNA probes. Labeled oligonucleotides may have a range of lengths. In some embodiments, labeled oligonucleotides have a length of at least 10, at least 11, at least 12, at least 13, at least 14, at least 15, at least 16, at least 17, at least 18, at least 19, or at least 20 nucleotides on each strand.

Oligonucleotides may be methylated at none, one, some, or all methylation sites (e.g., CpGs). In some embodiments, oligonucleotides comprise a fixed number of methylated nucleobases per molecule. In some embodiments, oligonucleotides comprise 0, 1, 2, 3, 4, 5, 6, 7, 8, 9, 10, 11, or 12 methylated nucleobases per molecule. In some embodiments, oligonucleotides comprise none, at least 1, at least 2, at least 3, at least 4, at least 5, at least 6, at least 7, at least 8, at least 9, at least 10, at least 11, or at least 12 methylated CpGs. For example, an oligonucleotide may comprise 9 CpG sites that are available for methylation but only 3 of those sites are methylated. In this example, the oligonucleotide has a methylation state of N3 (in general, “Nx” indicates an oligonucleotide in which x CpGs are methylated). In another example, an oligonucleotide of fixed length may comprise 9 CpG sites that are available for methylation and all 9 of those sites are methylated (N9). In some embodiments, oligonucleotides comprise 3 or 9 methylated CpGs. In some embodiments that comprise two different oligonucleotides, the oligonucleotides have different methylation states. In some embodiments, the second oligonucleotide has a greater number of methylation positions than the first oligonucleotide. In some embodiments, the first oligonucleotide is unmethylated and the second oligonucleotide is methylated. In some embodiments, the first oligonucleotide is hypomethylated and the second oligonucleotide is hypermethylated.

Oligonucleotides may comprise one or more types of methylation if more than one methylation site is present. In some embodiments, the methylation site is CpG. In some embodiments, the methylated CpG is chosen form 5-methylcytosine (5mC), 5-hydroxymethylcytosine (5-hmC), 5-formylcytosine (5-fC), or 5-carboxylcytosine (5-caC).

Oligonucleotides may be labeled for quantification. In some embodiments, labeled oligonucleotides are labeled with a fluorophore such as fluorescein (6-FAM or FAM), fluorescein dT, Cy (or Cy3™), TAMRA™, JOE, MAX, TET™, ROX, TYE™ 563, Yakima Yellow®, HEX, TEX 615, TYE™ 665, TYE 705, SUN, ATTO™ 488, ATTO™ 532, ATTO™ 550, ATTO™ 565, ATTO™ RHO101, ATTO™ 590, ATTO™ 633, ATTO™ 647N, Alexa Fluor® 488, Alexa Fluor® 532, Alexa Fluor® 546, Alexa Fluor® 594, Alexa Fluor® 647, Alexa Fluor® 660, Alexa Fluor® 750, 5′ IRDye® 700, 5′ IRDye® 800, 5′ IRDye® 800CW, Rhodamine Green™-X, Rhodamine Red™-X, 5-TAMRA™, Texas Red®-X, Lightcycler® 640, and Dy 750. Fluorescence measurements may be carried out using a microplate reader or any instrument deemed appropriate by one skilled in the art.

In some embodiments, labeled oligonucleotides of different methylation states are present in a sample, and each of these labeled oligonucleotides is labeled with a unique fluorescent tag, i.e., the labeled oligonucleotides of different methylation states are differentially labeled. For example, in an embodiment with two different labeled oligonucleotides, a first labeled oligonucleotide has a first fluorescent label and a second labeled oligonucleotide has a second fluorescent label. In some embodiments, the labeled oligonucleotides, which may be unmethylated or methylated, are separated into partitions, such as hypomethylated and hypermethylated partitions, before the oligonucleotides are quantified. In some embodiments, the labeled oligonucleotides, which may be unmethylated or methylated, are not separated into partitions prior to quantification. In some embodiments, the oligonucleotides are single-stranded or double-stranded.

Oligonucleotides may be incubated with MBDs or MBPs, in a buffer or partition, at various incubation times. In some embodiments, the incubation time is from 3 to 30 minutes, e.g., about 3, 5, 10, 20, or 30 minutes. In some embodiments, the incubation time is from 30 minutes to 2 hours, e.g., about 40, 50, 60, 90, or 120 minutes. In some embodiments, the incubation time is from 2 hours to 5 hours, or 5 hours to 8 hours, or 8 hours to 16 hours. In some embodiments, the incubation temperature is from 4° C. to room temperature, e.g., about 4° C., 18° C., or room temperature. In some embodiments, the incubation temperature is about 20° C., 21° C., 22° C., 23° C., 24° C., or 25° C. In some embodiments, shaking is applied during incubation.

### B. Partitioning Nucleic Acid Molecules; Methylated DNA-Binding Proteins

Disclosed methods herein comprise obtaining hypomethylated and hypermethylated partitions. In such methods, labeled oligonucleotides are physically partitioned based on binding to a methylated DNA-binding protein. In some embodiments, this approach can be used to assay the binding activity of a particular MBD or MBP for one or more labeled oligonucleotides, e.g., having different methylation states.

The methylated DNA-binding protein can be an affinity agent, such as an antibody with the desired specificity, a natural binding partner for methylated DNA, or a variant thereof (Bock et al., Nat Biotech 28:1106-1114 (2010); Song et al., Nat Biotech 29:68-72 (2011)), or artificial peptides selected e.g., by phage display to have specificity to a given target. In some embodiments, the modified nucleobase recognized by the agent is a modified cytosine, such as a methylcytosine (e.g., 5-methylcytosine). Where an antibody is used as the methylated DNA-binding protein, the oligonucleotides (e.g., methylated and/or unmethylated oligonucleotides) may be provided in single-stranded form. In some embodiments, the modified nucleobase recognized by the agent is a product of a procedure that affects the first nucleobase in the DNA differently from the second nucleobase in the DNA of the sample. Exemplary agents include methylated DNA binding domains (MBDs) and methylated DNA binding proteins (MBPs) as described herein, including proteins such as MeCP2, MBD1, MBD2, MBD4, or a methyl-CpG binding zinc finger protein, such as Kaiso.

Various levels of methylation can be partitioned using sequential elutions. For example, a hypomethylated partition (no methylation) can be separated from a methylated partition by contacting the nucleic acid population with the methylated DNA-binding protein. In some embodiments, the methylated DNA-binding protein is attached to magnetic beads (e.g., a methylated DNA-binding protein may be biotinylated and bound to streptavidin-coated beads). The beads are used to separate out the bound nucleic acids from the unbound nucleic acids. Subsequently, one or more elution steps are performed sequentially to elute nucleic acids, e.g., having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), an intermediately methylated partition (enriched in nucleic acids comprising low levels of methylation), and a hypermethylated partition (enriched in nucleic acids comprising high levels of methylation).

In some methods, nucleic acids bound to a methylated DNA-binding protein are subjected to a wash step. The wash step washes off nucleic acids weakly bound to the methylated DNA-binding protein. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification.

For further details regarding portioning nucleic acid samples based on characteristics such as methylation, see WO2018/119452, which is incorporated herein by reference.

In some embodiments, the partitioning is performed by contacting the nucleic acids with a methylated DNA binding domain (“MBD”) of a methylated DNA binding protein (“MBP”). In some such embodiments, the nucleic acids are contacted with an entire MBP. In some embodiments, an MBD binds to 5-methylcytosine (5mC), and an MBP comprises one or more MBDs. In some embodiments, the methylated DNA-binding protein is coupled to paramagnetic beads, such as Dynabeads® M-280 Streptavidin via a biotin linker. Partitioning into fractions with different extents of methylation can be performed by eluting fractions by increasing the salt concentration.

In some embodiments, bound nucleic acids are eluted by contacting the methylated DNA-binding protein with a protease, such as proteinase K. This may be performed instead of or in addition to elution steps using salt as discussed above.

Examples of methylated DNA-binding proteins contemplated herein include, but are not limited to: (a) the MBD family of proteins that preferentially bind to 5-methylcytosine over unmodified cytosine, i.e., MeCP2, MBD1, MBD2, MBD3, and MBD4; (b) RPL26, PRP8 and the DNA mismatch repair protein MHS6 preferentially bind to 5-hydroxymethylcytosine over unmodified cytosine; (c) FOXK1, FOXK2, FOXP1, FOXP4 and FOX13 preferably bind to 5-formyl-cytosine over unmodified cytosine (Iurlaro et al., Genome Biol. 14: R¹¹⁹ (2013)); (d) Antibodies specific to one or more methylated or modified nucleobases or conversion products thereof, such as 5mC, 5caC, or DHU; and (c) zinc finger proteins or zinc finger domains that bind methyl-CpG, e.g., Kaiso.

In general, elution is a function of the number of modifications, such as the number of methylated sites per nucleic acid molecule, with molecules having more methylation eluting under increased salt concentrations. To elute the nucleic acids into distinct populations based on the extent of methylation, one can use a series of elution buffers of increasing salt concentration. Salt concentration can range from about 100 nm to about 2500 mM. In some embodiments, the salt is sodium chloride, a sodium salt, potassium chloride, or a potassium salt. In some embodiments, the nucleic acids are released from the MBD or MBP after incubation for 5, 10, 15, 20, 25, or 30 minutes in the elution buffer.

In some embodiments, the method comprises one, two, three, or more partitions and each partition may comprise a salt concentration that is different from the other partitions. In some embodiments, a salt concentration is 0-0.5 M, 0.5-1.0 M, 1.0-1.5 M, 1.5-2.0 M, 2.5-3.0 M, 3.0-3.5 M. 0.1-1 M, 1.1-3.0 M, about 0.3 M, about 0.5 M, about 1.0 M, about 1.5 M, about 2.0 M, about 2.5 M, or about 3.0 M. In some embodiments, nucleic acid molecules are released from the MBD or MBP after incubation for 5, 10, 15, 20, 25, or 30 minutes in the partition.

In some embodiments, the process results in two or at least two partitions, such as a hypomethylated partition and a hypermethylated partition. In some embodiments, the process results in three (3) partitions, such as a hypomethylated partition, an intermediate partition, and a hypermethylated partition. Nucleic acids are contacted with a solution at a first salt concentration and comprising a molecule comprising an agent that recognizes a modified nucleobase, which molecule can be attached to a capture moiety, such as streptavidin. In some embodiments, the first salt concentration is from 0 M to 1 M, e.g., OM to 0.3 M, 0.3 M to 0.6 M, or 0.6 M to 1 M. At the first salt concentration a population of molecules will bind to the agent and a population will remain unbound. The unbound population can be separated as a “hypomethylated” population. For example, a first partition enriched in hypomethylated form of nucleic acids is that which remains unbound at a low salt concentration, e.g., 0 mM, 100 mM or 160 mM. Optionally, a partition enriched in intermediate methylated nucleic acids is eluted using an intermediate salt concentration, e.g., from 100 mM to 2000 mM (but higher than the first salt concentration). This is also separated from the sample. A partition enriched in hypermethylated form of DNA is eluted using a high salt concentration, e.g., from 0.6 M to 3 M or 1 M to 3 M (but higher than the intermediate salt concentration if used), e.g., from 2 M to 3 M.

In some embodiments, a monoclonal antibody raised against 5-methylcytidine (5mC) is used for partitioning. In some embodiments, a biotin-conjugated anti-5mC antibody is used for partitioning. If necessary, DNA is denatured, e.g., at 95° C. in order to yield single-stranded DNA fragments. Protein G coupled to standard or magnetic beads as well as washes following incubation with the anti-5mC antibody can be used to immunoprecipitate DNA bound to the antibody. Such DNA may then be eluted. Partitions may comprise unprecipitated DNA and one or more partitions eluted from the beads.

In some embodiments, the partitions of DNA are desalted and/or concentrated.

In some embodiments, results are compared to a reference standard. The reference standard can use a previously characterized protein, such as an MBD from a lot with known performance in the assay method. In some embodiments, the reference standard uses a biotin-conjugated anti-5mC antibody.

### C. Units of Binding for Methylated DNA Binding Domains/Proteins

Disclosed herein are methods for determining a quantitative unit of binding activity for an MBD or an MBP. As used herein, the terms “binding activity,” “binding unit,” “unit of binding,” “MBD,” “MBDx,” “MBDx unit of binding,” “MBDx binding unit” are synonymous and refer to an amount of protein (i.e., MBD or MBP) capable of binding a predetermined amount of a reference methylated oligonucleotide. In some embodiments, the protein is a methylated DNA binding domain (MBD). In some embodiments, the protein is a methylated DNA binding protein (MBP).

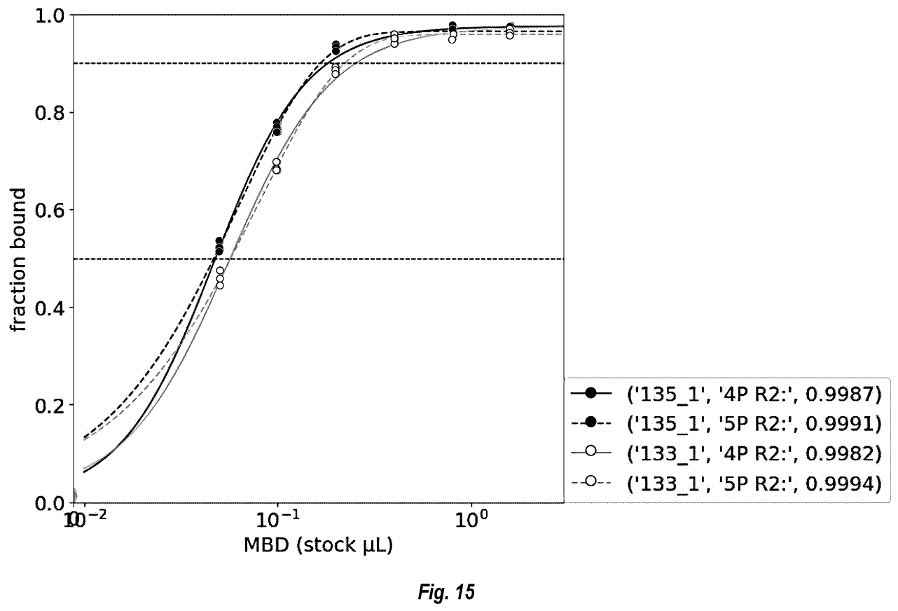

The binding unit is determined in units of amount or concentration of protein. It is understood by one skilled in the art that these binding units may vary depending on the reference oligonucleotide and the MBD or MBP used. In some embodiments, the reference oligonucleotide is labeled. In some embodiments, the reference oligonucleotide is single-stranded or double-stranded. In some embodiments, the methylated oligonucleotide comprises at least 2 methylated CpGs. In some embodiments, the reference methylated oligonucleotide comprises 2, 3, 4, 5, 6, 7, 8, 9, 10, 11, or 12 methylated CpGs. In some embodiments, the reference methylated oligonucleotide comprises 3 or 9 methylated CpGs. In some embodiments, the reference methylated oligonucleotide has a length of at least 10, at least 11, at least 12, at least 13, at least 14, at least 15, at least 16, at least 17, at least 18, at least 19, or at least 20 nucleotides on each strand. In some embodiments, the predetermined amount or concentration of a reference methylated oligonucleotide that is bound by the MBD or MBP is expressed as a fraction or as a percentage. In some embodiments, the predetermined amount ranges from at least 5% to 95% or at least 10% to 90% of the reference methylated oligonucleotide. In some embodiments, the predetermined amount or concentration ranges from at least 10% to 30%, at least 30% to 50%, at least 50% to 70%, or at least 70% to 90% of the reference methylated oligonucleotide. In some embodiments, the predetermined amount or concentration of oligonucleotide that is bound by the MBD or MBP is about 50%, 60%, 70%, 80%, or 90%. In some embodiments, the predetermined amount of oligonucleotide is incubated with a range of amounts or concentrations of MBD or MBP. By plotting the relationship between MBD concentration and fraction of bound oligonucleotide, a 4P or 5P binding curve can be fitted to the data (FIG. 15). In some embodiments, the MBDx unit of binding refers to the amount or the concentration of MBD or MBP required to bind to a predetermined amount of a reference methylated oligonucleotide. In some embodiments, the source of MBD or MBP in an assay is stable and/or replenishable. In some embodiments, “x” indicates the percentage of that predetermined amount of oligonucleotide. In some embodiments, “x” indicates the percentage of that predetermined concentration of oligonucleotide. For example, in some embodiments, “x” is 50 and “MBD50=1 μg,” which indicate that 50% of a reference methylated oligonucleotide is bound by 1 microgram of protein (i.e., MBD or MBP). In some embodiments, “x” is 90 and “MBD90=1 μg,” which indicate that 90% of a reference methylated oligonucleotide is bound by 1 microgram of protein (i.e., MBD or MBP). In some embodiments, the “x” in MBDx is 40, 60, 70, or 80, which indicates that about 40%, 60%, 70%, or 80%, of the reference methylated oligonucleotide is bound by the protein. A higher MBDx value indicates that more protein is required to reach the same level of oligonucleotide binding. For example, if MBD50=10 μg for a first protein and MBD50=1 μg for a second protein, these values indicate that the first protein requires 10 times more protein than the second protein to achieve 50% binding of the reference methylated oligonucleotide. In some embodiments, the amount or concentration of protein is a mass or mass per unit volume, respectively. In some embodiments, the amount or concentration of protein is a molar quantity or moles per unit volume (e.g., molarity), respectively.

### III. Kits

Also provided are kits comprising the compositions as described herein. The kits can be for use in performing the methods as described herein. In some embodiments, a kit comprises a plurality of labeled oligonucleotides. In some embodiments, the plurality of labeled oligonucleotides comprises or consists of probes comprising a capture moiety that hybridize to target regions having a type-specific epigenetic variation and a copy number variation. In some embodiments, the kit comprises a solid support linked to a binding partner of the capture moiety. In some embodiments, the kit comprises adapters. In some embodiments, the kit comprises PCR primers that anneal to an adapter. In some embodiments, the kit comprises additional elements elsewhere herein. In some embodiments, the kit comprises instructions for performing a method described herein.

In some embodiments, a kit further comprises an agent that recognizes methylcytosine in DNA. In some such embodiments, the agent is an antibody or a methyl binding protein or methyl binding domain. In some embodiments, the kit comprises labeled oligonucleotides that specifically bind to sequence-variable target region sets. In some such embodiments, the labeled oligonucleotides comprise a capture moiety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

The following examples describe development of fluorescence-based assays for analyzing methylated DNA binding domains (MBDs) or methylated DNA binding proteins (MBPs), which are herein termed MBD assays. The assays may be used to assess the performance of protein-based enrichment of methylated DNA.

### Example 1. Materials and Methods

### A. A Bead-based MBD Fluorescence Assay

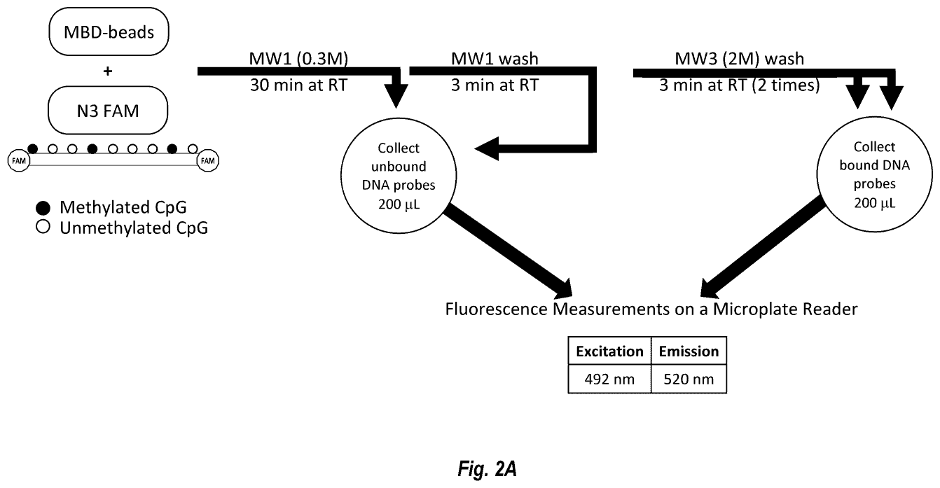

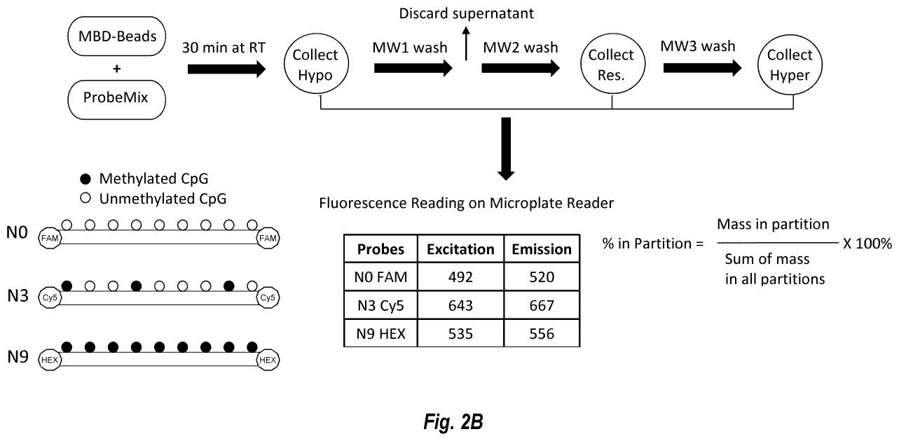

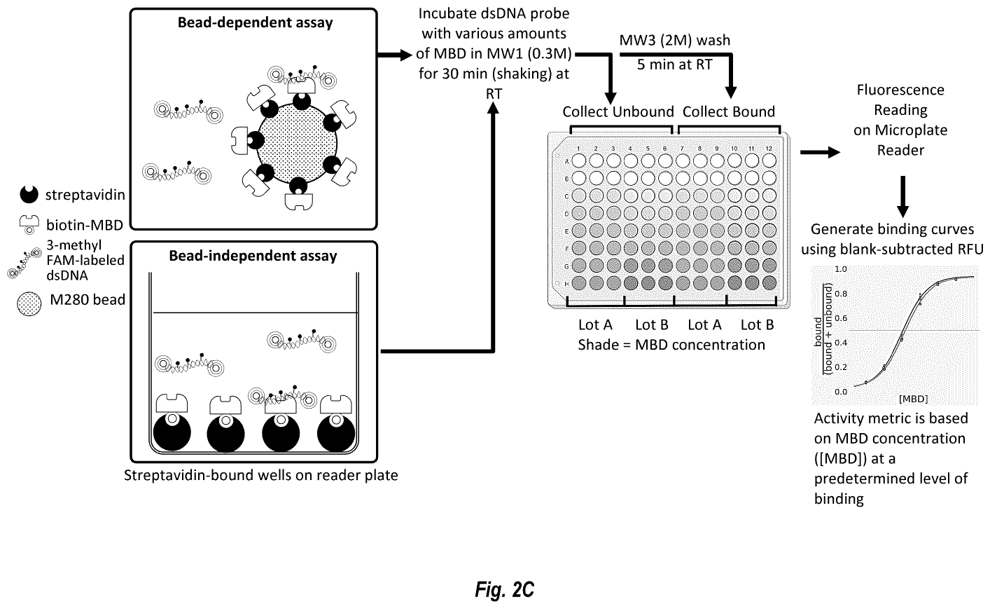

FIG. 2A shows the workflow used in the bead-based MBD assay of this example to group DNA probes with different methylated states into hypomethylated and hypermethylated partitions. These partitions are used to assay the binding affinity of an MBD to a FAM-labeled dsDNA probe that is 150 nucleotides in length, having 3 methylated CpGs (N3 probe).

MBPs were conjugated to Dynabeads™ M-280 Streptavidin (Thermo Fisher Scientific, Catalog No: 11205D) according to manufacturer recommendations. The MBDs conjugated to Dynabeads™ (MBD-beads) were incubated with N3 FAM-labeled DNA probes in MW1, a solution containing 0.3 M NaCl, for 30 minutes at room temperature. After applying a magnetic source, 100 μL of the supernatant was collected, containing probes not captured by MBD-beads. The MBD-beads were then washed with MW1 for 3 minutes at room temperature. After applying a magnetic source, the wash was collected as well. The initial supernatant and wash together formed the hypomethylated partition. After that, the MBD-beads were washed twice with MW3, a solution containing 2 M NaCl, and 100 μL of the supernatant, i.e., the hypermethylated partition, was collected from each wash (i.e., a total of 200 μL was collected). Fluorescence intensity of the MW1 (hypo) partition and MW3 (hyper) partition were measured using a microplate reader (Spark). Mean relative fluorescence units (RFU) were recorded. The amount of N3 probe in the hypomethylated partition and the hypermethylated partition were calculated using a standard curve. The amount of DNA probes present in a partition is determined by correlating the measured RFU value for said partition to ng of DNA probes using the DNA probe standard curves (described below in Example 1.B). Alternatively, the RFU values may be used without conversion to ng of probe; for example, the RFU values may be subjected to background subtraction and then compared directly.

### B. DNA Probe Standards for Establishing Standard Curves

Standard curves were used to establish accuracy and precision of the MBD assay. Sets of N3 probe standards were prepared in triplicate using a serial dilution factor of 2×. Each set included standards at the following probe concentrations: 0.52, 0.26, 0.13, 0.065, 0.0325, and 0.01625 ng/μL, and a blank standard with no probe. Fluorescence intensity of the DNA probe standards was assayed in MW1 and MW3 buffers and mean RFU measurements were recorded. The RFU of the blank was subtracted from each RFU measurement. A standard curve was generated by linear regression, linear regression with the y-intercept forced through 0, and regression with log-log transformation. Linear regression with the y-intercept forced through 0 gave the highest R² values (R²>0.995 for all experiments).

### C. A Unit of Binding as a Measure of Binding Affinity of Methylated DNA Binding Domains (MBDs) for DNA Probes

A quantitative unit of binding for MBDs was developed to facilitate quantification of binding variability of MBDs across different lots. This section describes the determination of the amount of a given preparation of MBD needed to bind 50% of DNA probes in a sample. The unit of binding in this example is termed MBD50.

The unit MBD50 is an amount of MBP that binds 50% of the DNA probes in a 200 μL sample containing 13 ng of N3 probes under assay conditions described herein.

N3 probes were used in the MBD assay described in Example 1.A. DNA probes that were bound by MBDs in MW1 were released from the MBDs by treatment with MW3, and thereby recovered for quantitation by fluorescence. The fraction of bound DNA probes was calculated by dividing the amount of recovered DNA probes (i.e., ng of eluted or recovered DNA probes in MW3 that correlates with measured RFU value) by the total amount of DNA probes used in the assay. (The total amount of a particular DNA probe is the sum of (1) the amount of probes in MW1 (i.e., unbound DNA probes) and (2) the amount of probes in MW3 (i.e., bound DNA probes).

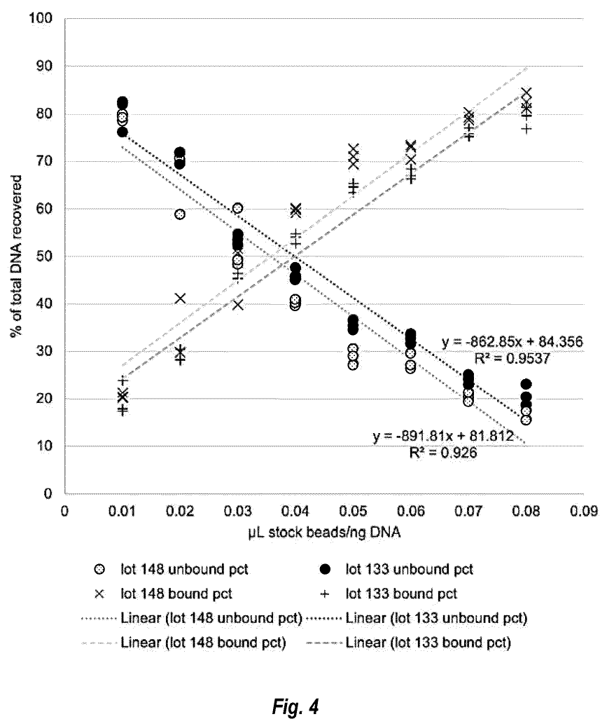

A graph was generated of the percentage of total DNA recovered as a function of the amount of MBD-beads (μL) added per well. A binding curve (as shown in FIG. 4) was generated, from which MBD50 was derived.

13 ng of N3-FAM DNA probes were used. MBD beads were used at 0.01-0.08 μL stock beads per ng probe. Two lots of MBD were tested (lot 133 and lot 148). Results are shown in FIG. 4. From these data, it was determined that 0.0398 μL of stock beads/ng probe provided 1 MBD50 unit for lot 133 and 0.0357 μL of stock beads/ng probe provided 1 MBD50 unit for lot 148. The amounts corresponding to 1 MBD50 unit were determined by linear regression of the data shown in FIG. 4.

### D. A Plate-Based (Bead-Independent) MBD Assay

A plate-based, i.e., bead-independent, MBD assay was also developed. Instead of beads, the MBDs were adhered to the surface of microplate wells. This was done to eliminate the influence of the quality of the streptavidin beads (to which MBD is bound) on the outcome of the assay. This version of the assay is simpler and automation-friendly, without requiring streptavidin beads clean-up, magnetic beads separation, and a separate fluorescent plate. The bead-based assay may still be used to assess the quality of streptavidin beads in conjunction with the MBD-biotin protein.

All wells of a streptavidin-coated plate (Thermo Scientific Cat #15503) were washed three times with 200 μL of MB buffer (i.e., the bind/wash buffer supplied as MethylMiner™ 5× bind/wash buffer, Life Tech Cat. #A42947). The MBD reagent was prepared as follows: 28 μL of biotin-labeled MBPs was diluted into with 772 μL of MB buffer. 5-40 μL of MBD reagent was pipetted per well of columns 1-6 (C1-C6) of the plate (see Table 1) to give the volume of undiluted MBP corresponding to the values per 13 ng DNA probe shown in Table 1 upon removal of supernatant and addition of 100 μL of N3 FAM DNA probe in MW1 at 0.13 ng/u L. By trapping all biotin-conjugated MBD proteins with streptavidin coated in excess (Binding Capacity of the plate according to its product insert: ˜ 125 pmol D-biotin/well), lot-to-lot difference of the plate should be minimized. 100 μL of MB buffer was added to standard columns C8 and C9.

TABLE 1
Plate layout.

| Volume of MBP | | | | | | |
|---|---|---|---|---|---|---|
| per 13 ng DNA | | | | | | |
| probe | | C1 | C2 | C3 | C4 | C5 |
| 0.175 | A | Bound_MW1 | Bound_MW3 | Bound_MW3 | Unbound_MW1 | Unbound_MW1 |
| 0.35 | B | | | | | |
| 0.525 | C | | | | | |
| 0.7 | D | | | | | |
| 0.875 | E | | | | | |
| 1.05 | F | | | | | |
| 1.225 | G | | | | | |
| 1.4 | H | | | | | |

| Volume of MBP | | | | | | Standard |
|---|---|---|---|---|---|---|
| per 13 ng DNA | | | | | | concentration |
| probe | | C6 | C7 | C8 | C9 | of MBP |
| 0.175 | A | Unbound_MW1 | Bound_MW3T | Std_MW1 | Std_MW3 | 260 |
| 0.35 | B | | | | | 130 |
| 0.525 | C | | | | | 65 |
| 0.7 | D | | | | | 32.5 |
| 0.875 | E | | | | | 16.25 |
| 1.05 | F | | | | | 8.125 |
| 1.225 | G | | | | | 4.0625 |
| 1.4 | H | | | | | Blank |

The microplate was incubated for 2 hours at room temperature with shaking. Supernatant was removed and every well was then washed three times with MB buffer. 100 μL of 0.13 ng/μL N3 FAM DNA probe in MW1 was added to the wells from C1 to C3. The plate was incubated for 30 minutes with shaking.

From wells A-H of Column 1 (C1; Bound_MW1), after the incubation step described above, 100 μL of the sample supernatant was transferred to Column 4. This step transferred from C1 to C4 the DNA probes not bound to MBPs. Then, 100 μL of MW1 was added to C1 and the bound DNA probes were presumed to still be attached to the MBPs.

From wells A-H, of Column 2 (C2; Bound_MW3), after the incubation step described above, 100 μL of the sample supernatant was transferred to C5. This step transferred from C2 to C5 the DNA probes not bound to MBPs. Then, 100 μL MW3 was added to C2 and the plate was incubated for 5 minutes with shaking. DNA probes that were bound by the MBPs were released.

From wells A-H of Column 3 (C3; Bound_MW3), after the incubation step described above, 100 μL of the sample supernatant was transferred to C6. This step transferred DNA from C3 to C6 the DNA probes not bound to MBPs. Then, 100 μL MW3 was added to C3 and the plate was incubated for 5 minutes with shaking. This incubation step was carried out concurrently with the MW3, 5-minute incubation step described for C2. DNA probes that were bound by the MBPs were released and 100 μL of the supernatant was transferred to C7.

Wells A4-H4, i.e., Column 4 (C4; Unbound_MW1) contained unbound DNA probes in MW1 and that were transferred from C1.

Wells A5-H5, i.e., Column 5 (C5; Unbound_MW1) contained unbound DNA probes in MW1 and that were transferred from C2.

Wells A6-H6, i.e., Column 6 (C6; Unbound_MW1) contained unbound DNA probes in MW1 and that were transferred from C3.

Wells A7-H7, i.e., Column 7 (C7; Bound_MW3T) contained bound DNA probes in MW3 and that were transferred here from C3.

Wells A8-H8, i.e., Column 8 (C8; Std_MW1) were used for DNA probe standards in MW1. Wells A9-H9, i.e., Column 9 (C9; Std_MW3) were used for DNA probe standards in MW3.

The microplate was centrifuged at 3,900 rpm for 3 minutes, after which fluorescence measurements were recorded using a plate reader such as Spark. The excitation and emission wavelengths used for the N3 FAM DNA probes were 492 nm and 520 nm, respectively.

### E. Alternative Plate-Based Assay Parameters

The plate-based MBD assay was carried out as described above in Example 1.D with the following modifications.

### F. Plate Layout

A microplate with columns 1-12 and rows A-H were labeled as shown in Table 2. Incubation times, the number of wash steps, and the amount of DNA probes are shown.

TABLE 2
Plate layout.

| | C1 | C2 | C3 | C4 | C5 | C6 | C7 | C8 | C9 | C10 | C11 | C12 |
|---|---|---|---|---|---|---|---|---|---|---|---|---|
| A | 2 hrs, | 2 hrs, | 2 hrs, | 2 hrs, | 1 hr, | 0.5 hr, | 2 hrs, | 2 hrs, | 2 hrs, | 2 hrs, | 1 hrs, | 0.5 hrs, |
| B | 3X, | 3X, | 3X, | 1X, | 3X, | 3X, | 3X, | 3X, | 3X, | 3X, | 3X, | 3X, |
| C | 52 ng | 52 ng | 13 ng | 13 ng | 13 ng | 13 ng | 52 ng | 26 ng | 13 ng | 13 ng | 13 ng | 13 ng |
| D | | | | | | | | | | | | |
| E | | | | | | | | | | | | |
| F | | | | | | | | | | | | |
| G | | | | | | | | | | | | |
| H | | | | | | | | | | | | |

Unbound in MW1
Bound in MW3

### G. Wash Steps

Previously, before and after incubation with MBPs, the microplate was washed three times with MB buffer. The wash steps, particularly after incubation with MBPs, removed excess MBPs that were not captured by streptavidin. In this example, the effect of three washes (3×) with MB buffer was compared to the effect of only one wash step (1×).

### H. Protein Incubation Time

Previously, a 2-hour incubation period was used for streptavidin to capture the MBPs. In this example, incubation times of less than 2 hours were evaluated.

### I. DNA Probe Amounts

Previously, in the beads-based MBD assay (described in Example 1.A), 13 ng of N3 FAM DNA probes were used. In this example, larger amounts of N3 FAM DNA probes, i.e., 26 ng and 52 ng, were evaluated.

### J. Optimization of DNA Probe Incubation Time

The plate-based MBD assay was carried out as described above in Example 1.D with the following modification—the DNA probe incubation time ranged from 30 minutes to 5 hours. Longer incubation times, such as 8-16 hours or overnight at 4° C. were also considered. A microplate with columns 1-12 and rows A-H were labeled as shown in Table 3. The volumes of MBPs used were adjusted such that the 50% fraction point would appear in the center of each graph showing RFU fraction for bound and unbound DNA probes.

TABLE 3
Plate Lot No. VL308272.

| | C1 | C2 | C3 | C4 | C5 | C6 | C7 | C8 | C9 | C10 | C11 | C12 |
|---|---|---|---|---|---|---|---|---|---|---|---|---|
| A | 0.5 hr | 1 hr | 2 hrs | 3 hrs | 4 hrs | 5 hrs | 0.5 hr | 1 hr | 2 hrs | 3 hrs | 4 hrs | 5 hrs |
| B | | | | | | | | | | | | |
| C | | | | | | | | | | | | |
| D | | | | | | | | | | | | |
| E | | | | | | | | | | | | |
| F | | | | | | | | | | | | |
| G | | | | | | | | | | | | |
| H | | | | | | | | | | | | |

Unbound in MW1
Bound in MW3

### K. Incubation Temperature

The plate-based MBD assay was conducted at 18° C., 24° C., and 25° C. while the remaining assay parameters were kept the same as described in Example 1.D. MBD-protein lot 135 was used. MBD-protein with 2× freeze-thaw cycles (after storage at −80° C.) were used.

L. Bead-based MBD Assay with a Triplex Probe Mix

This example describes the MBD assay with a triplex probe mix. The triplex probe mix comprised 50 ng of each of the following DNA probes: NO-FAM, N3-Cy5, and N9-HEX. The assay methods were as described in the Examples 1.A.-D. above with modifications.

The wash steps were modified as follows. First, the volume of supernatant from the hypomethylated partition was 100 μL instead of 200 μL. Second, after the hypomethylated partition, 2 residual wash steps, MW2, were introduced. The volume of each residual wash step was 100 μL and the two washes were collected in the same well. Third, the hypermethylated partition comprised 2 washes at 100 μL each, which were also collected in the same well.

Five different lots of MBPs were tested. These lots were labeled lot4 (which had been previously identified as having lower performance), test1, test2, test3, and test4 (each of test1-4 were being newly tested). A lot with known good quality MBP was used as the control lot.

### Example 2. Accuracy of the MBD Assay

The materials and methods described in Example 1 were used to determine the accuracy of the MBD assay. Three rounds of the assay (i.e., Experiment (Exp.) 3, Exp. 4, and Exp. 5 in Tables 4 and 5) were carried out with modifications at each subsequent round to improve the linearity and limit of quantitation of the standard curves.

TABLE 4
R² of Standard Curves.

| | | R² | | |
|---|---|---|---|---|
| | | Linear | Linear | Log |
| Buffer | Experiment | y = mx | y = mx + b | y = mx + b |
| MW1 | 3 | 0.9979 | 0.9970 | 0.9869 |
| MW1 | 4 | 0.9992 | 0.9986 | 0.9985 |
| MW1 | 5 | 0.9997 | 0.9997 | 0.9970 |
| MW3 | 3 | 0.9952 | 0.9936 | 0.9940 |
| MW3 | 4 | 0.9996 | 0.9994 | 0.9991 |
| MW3 | 5 | 0.9989 | 0.9987 | 0.8990 |

TABLE 5
Back-calculated ng/μL and % Recovery of DNA Probes.

| Standards | MW1 | | | MW3 | | |
|---|---|---|---|---|---|---|
| ng/μL | Exp. 3 | Exp. 4 | Exp. 5 | Exp. 3 | Exp. 4 | Exp. 5 |
| 0.52 | 99.6 | 99.96 | 99.46 | 97.85 | 100.32 | 101.11 |
| 0.26 | 97.55 | 100.67 | 102.85 | 99.54 | 99.82 | 95.21 |
| 0.13 | 97.38 | 99.38 | 97.51 | 122.39 | 96.67 | 98.48 |
| 0.065 | 105.88 | 90.8 | 97.89 | 109.59 | 94.59 | 96.56 |
| 0.0325 | 115.37 | 90.28 | 95.37 | 109.15 | 93.51 | 130.96 |
| 0.0163 | 125.74 | 86.79 | 98.17 | 133.19 | 92.54 | 98.98 |
| 0.0081 | 125.42 | | 113.86 | 134.09 | | 108.69 |
| 0.0041 | 155.62 | | 116.6 | 145.2 | | 106.82 |

Parameters that were modified and/or optimized included varying (1) the number of MBP standards to improve limit of quantitation (LOQ) range, (2) the amount of reagents used for improved % coefficient variant (% CV), and (3) the MBD-bead: N3 ratio and step size to maximize linear range of the assay curves, (4) mixing/incubation times of unbound and wash partitions before measurement. An additional step of cleaning the assay plate was also tested. As shown in Tables 4 and 5 for Exp 3 through Exp 5, the protocol modifications that were introduced improved the linearity of the standard curves and limit of quantitation (LOQ).

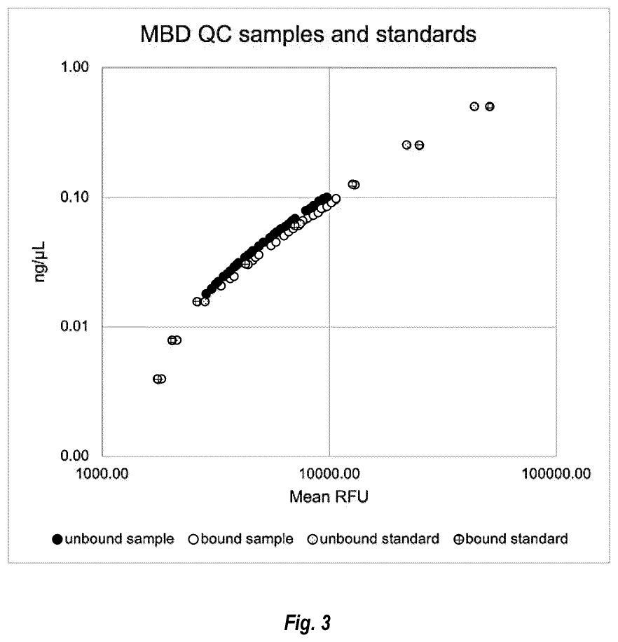

Three curve-fitting algorithms were tested to provide the best standard curve fit and their R² values for are shown above in Table 4. While forcing of the intercept through 0 resulted in slightly higher R² value for both MW1 and MW3, log-log transformation did not improve the fit of the curve. The standard curves generated met the limit of quantitation (LOQ) and % coefficient of variation (% CV) criteria of <20%. In Exp 5, MBP standards down to 0.0041 ng/μL met the LOQ % recovery criteria of 80-120% (Table 5). In conclusion, all samples and standards that were measured were within the detection range (i.e., the LOQ) of the assay (FIG. 3 and Table 6).

TABLE 6
Quality Control (QC) Standards and Samples.

| | |
|---|---|
| MW1 | MW3 |

| | Back- | | Back- | |
|---|---|---|---|---|
| Standards | calculated | % | calculated | % |
| ng/μL | ng/μL | Recovery | ng/μL | Recovery |
| 0.5000 | 0.4985 | 100 | 0.5838 | 117 |
| 0.2500 | 0.2434 | 97 | 0.2783 | 111 |
| 0.1250 | 0.1369 | 109 | 0.1371 | 110 |
| 0.0625 | 0.0700 | 112 | 0.0684 | 110 |
| 0.0313 | 0.0354 | 113 | 0.0363 | 116 |
| 0.0156 | 0.0174 | 111 | 0.0163 | 104 |
| 0.0078 | 0.0089 | 113 | 0.0092 | 117 |
| 0.0039 | 0.0052 | 134 | 0.0060 | 153 |

### Example 3. MBD Binding Affinity and Unit of Binding, MBD50

Binding affinity of MBP for the DNA probes, as expressed by the term MBD50, was assessed as described above in Example 1.C. Two lots of MBPs, Lots 133 and 148, were used for comparison (FIG. 4). MBD50 was determined by solving for x when y=50% and multiplying x by 100. R², x, and y values using MBPs from both Lots 133 and 148 are shown below in Table 7.

TABLE 7
Stock Bead Concentration at 50% of DNA Recovered.

| Lot No. | R² | y | x when y = 50% |
|---|---|---|---|
| 133 | 0.954 | −862.85x + 84.356 | 0.0398 |
| 148 | 0.926 | −891.81x + 81.812 | 0.0357 |

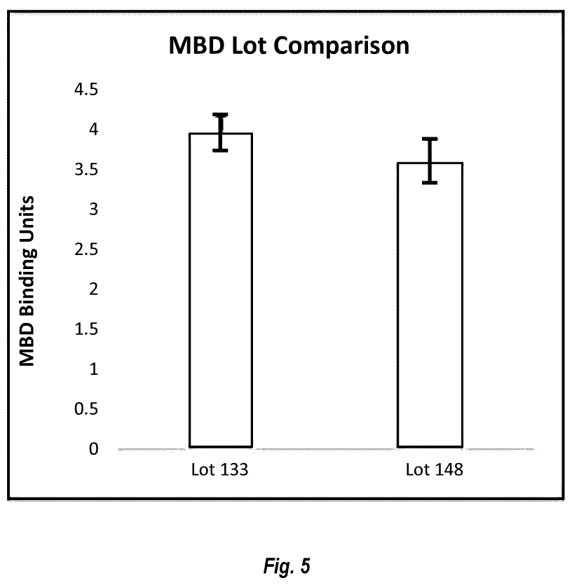

As shown below in Table 8 and FIG. 5, the MBD50s for Lot 133 and Lot 148 are 3.98 and 3.57, respectively.

TABLE 8
MBD Unit of Binding.

| μL stock | μL stock | MBD50 unit |
|---|---|---|
| beads/200 ng | beads/ng DNA | of binding |
| 2 | 0.01 | 1 |
| Lot 148 50% bound: | 0.0357 | 3.57 |
| Lot 133 50% bound: | 0.0398 | 3.98 |

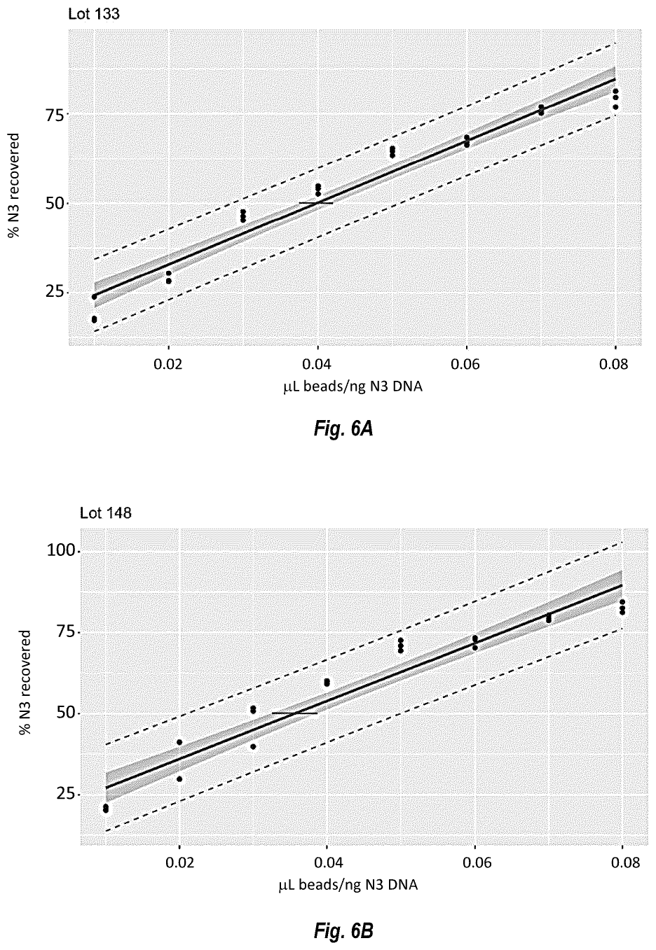

Precision of the MBD assay was determined using standard curves as described above in Example 1.B. FIGS. 6A-B show the % of N3 FAM-labeled DNA probes that were recovered using varying ratios of MBD-beads to DNA probes. The values for the μL of MBD-beads for each ng of N3 FAM-labeled DNA (μL/ng) that showed 50% for both lots were calculated. For Lot 133, the value was 0.04005709 with a lower bound of 0.03782419 and an upper bound of 0.04228999. For Lot 148, the value was 0.03636149 with a lower bound of 0.03340213 and an upper bound of 0.03932086. The confidence intervals at 50% for both Lots 133 and 148 were 95%.

### Example 4. A Plate-Based (Bead-Independent) MBD Assay

This example describes a plate-based MBD assay that does not require the use of streptavidin beads. An MBD assay that is dependent on streptavidin beads may be influenced by the quality of the streptavidin beads as well as the quality of the MBP conjugated via biotin to the streptavidin beads. An MBD assay that is independent of streptavidin beads would provide results that more directly reflect the properties of the MBD. By replacing streptavidin beads with streptavidin plates, the MBD assay can directly assay the properties of the MBPs.

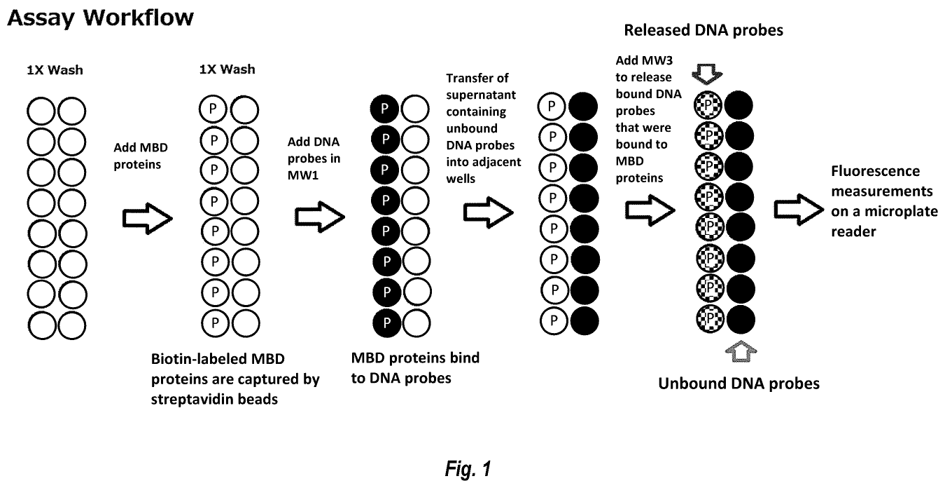

The assay was improved by the removal of several method steps, such as magnetic streptavidin beads clean-up, magnetic streptavidin beads separation, and the transfer of beads into a microplate for fluorescence measurements. The workflow for this assay is shown in FIG. 1. The materials and methods used in this example were described above in Examples 1.D-E.

A. Yield Fraction Graphs Generated from Relative Fluorescence Units (RFUs)

A standard curve is typically used to convert a fluorescence signal into a concentration or mass unit. If analytes are in different matrices, a matrix effect may be observed; a matrix effect is the combined effect of all components in the sample, other than the analyte, on the measurement of the quantity. A matrix effect can be addressed by generating a standard curve for each matrix.

The MBD assay described herein is used, not to measure analyte concentrations, but to provide fractions of unbound and bound DNA probes that are in two different matrices, MW1 and MW3. The unbound DNA probes are in MW1 while the bound DNA probes are in MW3. Therefore, standard curves are needed only if the fluorescent values for the standard solutions in the two matrices are different.

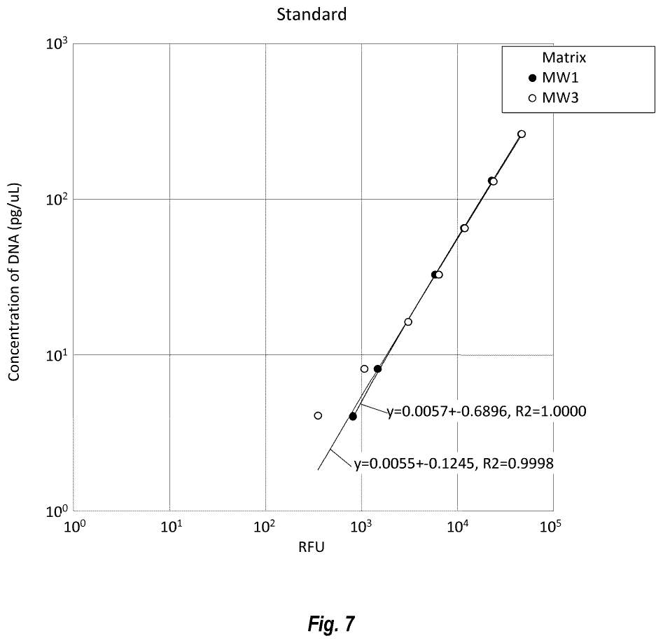

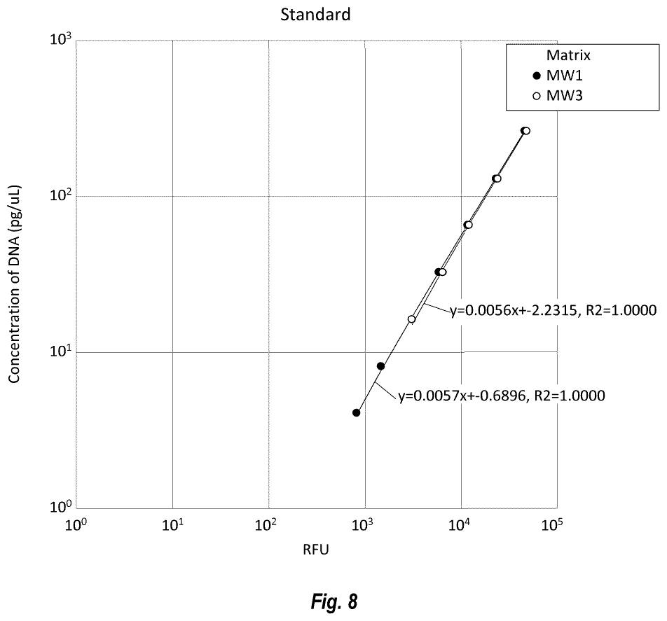

Standard curves for the plate-based MBD assay were prepared. In the standard curves shown in FIG. 7, a matrix effect was observed for two low concentration points for the MW3 solution. These two points were removed to produce the standard curves shown in FIG. 8. In this figure, both standard curves overlapped with a linearity of 1, indicating that the matrix effect was negligible.

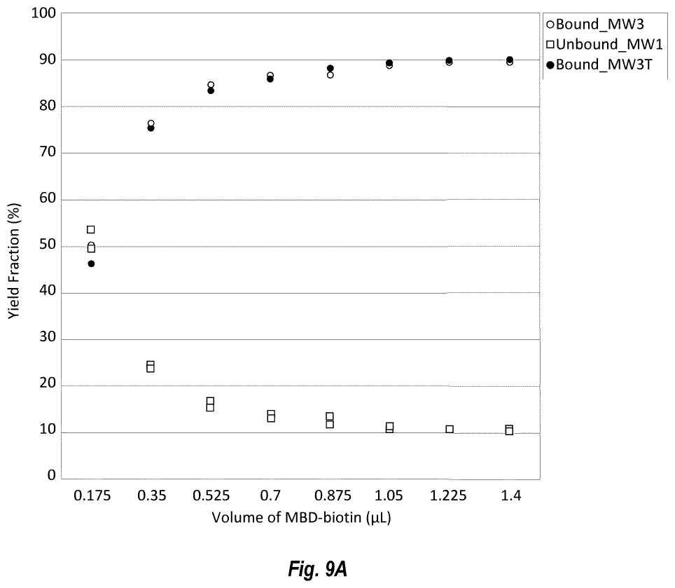

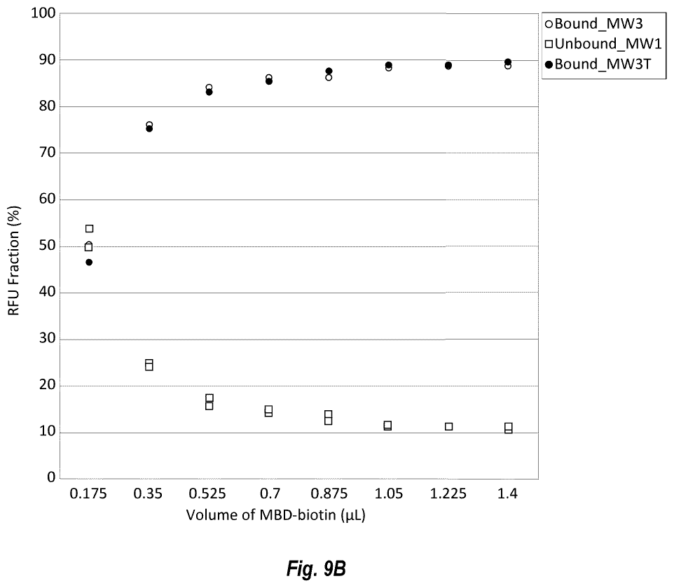

Because no significant matrix effect was observed, it should not be necessary to use standard curves. Rather, the fraction curves may be generated by using the relative fluorescence units (RFU) directly. To test this, a yield fraction graph was generated using standard curves (FIG. 9A) and an RFU fraction graph was generated without standard curves but using measured RFU instead (FIG. 9B). As shown in FIGS. 9A-B, the yield fraction graph and the RFU fraction graph are equivalent.

The data point for Bound_MW1 (C1) was excluded from the yield and RFU fraction graphs (FIGS. 9A-B) because its fluorescence signal was low. This low signal suggests that bound DNA probes in MW3 need to be released from the MBPs before fluorescence signal is measured.

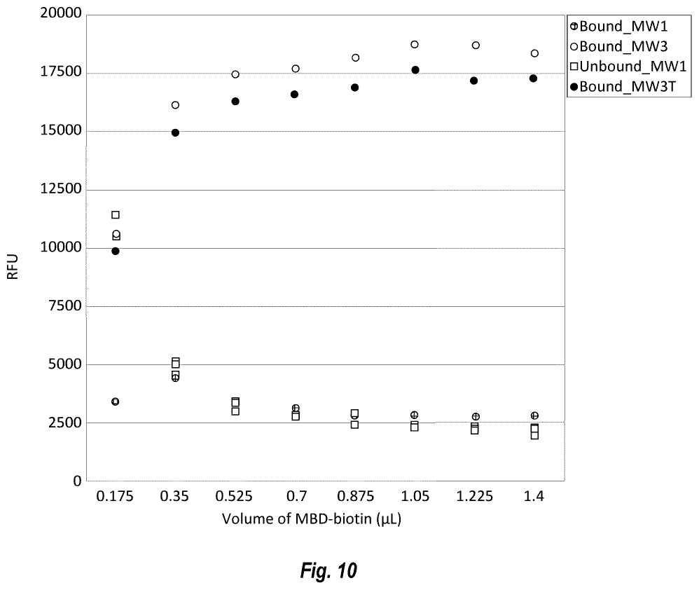

Low fluorescence signal was also observed for Bound_MW3T (C7) when compared to Bound_MW3 (C2) in the RFU graphs (FIG. 10). The reduction was caused by the small fraction of DNA probes which were still attached to the MBPs even after the addition of MW2 and not transferred to C7, or by the absence of MBPs on the well surfaces of C7. In either case, the difference was insignificant as the fraction curves for Bound_MW3 and Bound_MW3T overlapped. Therefore, the extra step of transferring bound DNA probes into empty wells could be omitted.

### B. Wash Steps, Protein Incubation Time, and DNA Probe Amounts

In this example, the wash steps, protein incubation time, and the DNA probe amounts in the plate-based MBD assay were evaluated.

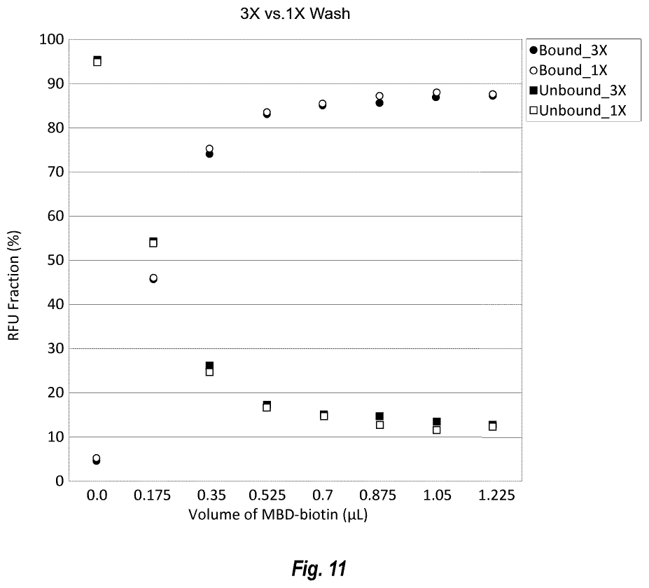

FIG. 11 compares the effect of a single wash step to a triple wash step. Because a single wash was found to be as effective as a triple wash, the triple wash was replaced by a single wash. Although the RFU values and the RFU fractions shown were not adjusted with an RFU for a blank, the trend of each curve was not significantly affected.

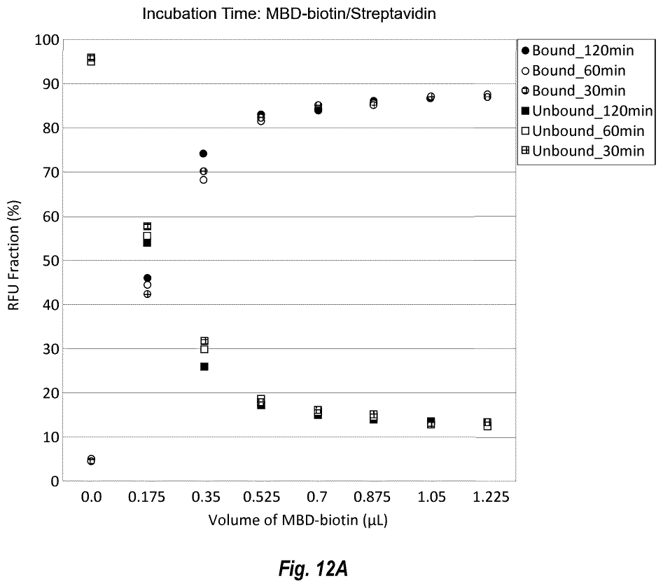

As shown in FIGS. 12A-B, a decrease in protein incubation time caused the 50% fraction point to shift to the right. This indicates that there were lower amounts of bound DNA probes with shorter incubation times. Therefore, the 2-hour incubation time in the method was unchanged. Although the RFU values and the RFU fractions shown were not adjusted with an RFU for a blank, the trend of each curve was not significantly affected.

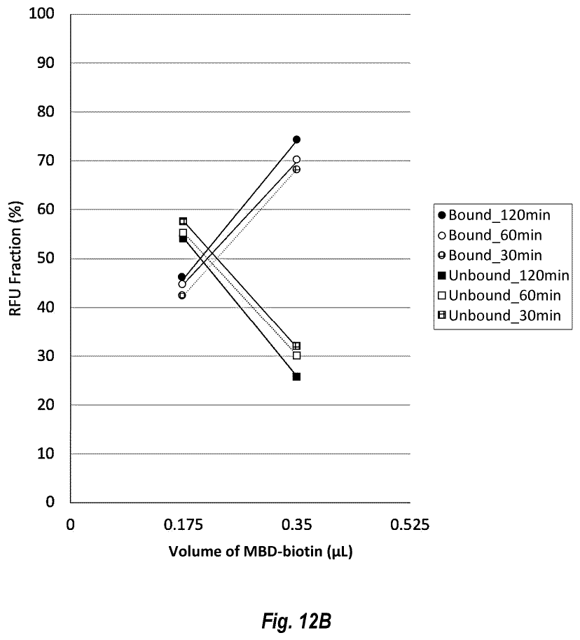

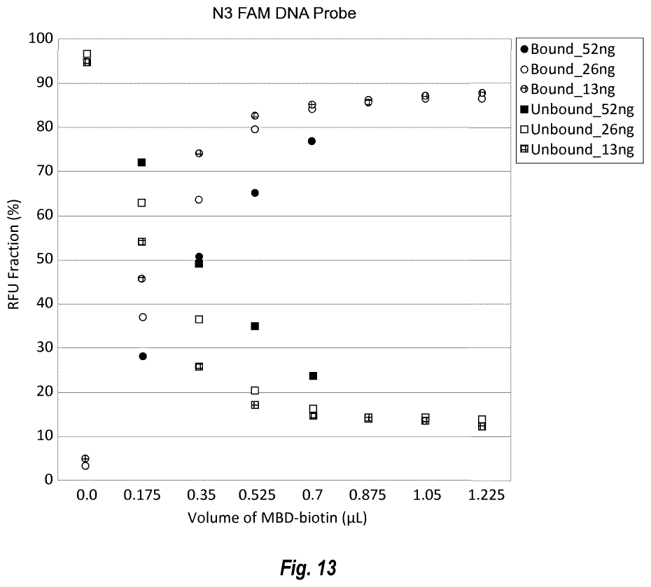

As shown in FIG. 13, increasing amounts of N3 FAM DNA probe caused the 50% fraction point to shift to the right due to an excess of unbound DNA probes. Some data points for 52 ng DNA probe were not recorded because the fluorescence signals were too high and beyond the detection range of the plate reader used. Data points for 13 ng of DNA probe were comparable to those for 26 ng, and had less background noise. It was also considered favorable to use lower amounts of reagents in an assay. In some methods, 13 ng of DNA probes was used. In other methods, 20 ng of DNA probes was used.

### C. DNA Probe Incubation Time

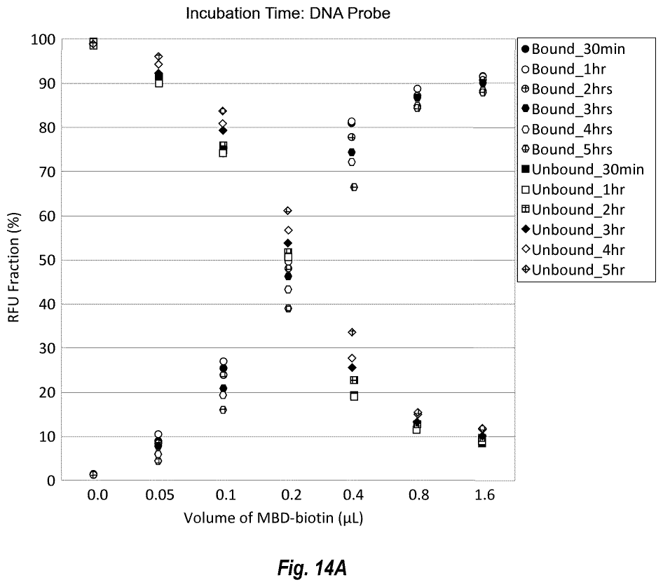

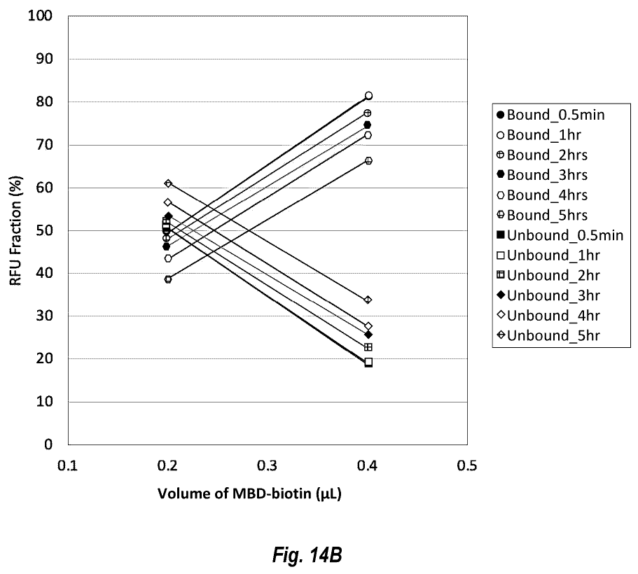

In this example, longer times for DNA probe incubation in MW1 buffer conditions were evaluated as longer times may help more DNA probes bind to MBD-proteins. As shown in FIGS. 14A-B, times up to 1 hour did not change the 50% fraction point. These experiments used 13 ng DNA probes. However, times of 2 hours and more caused the 50% fraction point to shift to the right, indicating that were more unbound DNA probes than bound DNA probes when longer incubation times were used. This may be due to the MBPs losing their binding affinity to the DNA probes after incubation at room temperature for longer than 1 hour. Therefore, 30 minutes was considered an appropriate incubation time for MW1 when using 13 ng DNA probes. In other experiments, 60 minutes gave good performance when using 20 ng DNA probes and LSE buffer (containing 0 M NaCl) (data not shown).

### D. Quality of MW1 Buffer on DNA Probe Binding to MBD

In this example, the effect of changes in salt concentration in MW1 on the binding of N3 FAM DNA probe to MBD was evaluated. Several rounds of the assay had shown that while the volume of MBD-biotin at 50% yield fraction satisfied the % CV criteria of <20% (Table 9), intermediate precision (% CV) and operator-to-operator variability (Table 10) could be improved. In reviewing precision and variability of 10 runs by 3 different operators, the intermediate precision for operator 3 alone was 26.0% (Table 10), which was too high and indicative of poor precision. For the experiments in Table 10, all operators used the same lot of high salt elution buffer. Operators 1 and 2 used the same lot of low salt elution buffer and operator 3 used a different lot of low salt elution buffer.

TABLE 9
Volume of MBD-biotin (μL) at 50% Yield Fraction.

| | | | | | | | Mean | |
|---|---|---|---|---|---|---|---|---|
| | Tube or Replicate | | | | | | (μL; | % |
| | 1 | 2 | 3 | 4 | 5 | 6 | n = 6) | CV |
| Tube-to-tube | 0.292 | 0.272 | 0.272 | 0.275 | 0.274 | 0.259 | 0.274 | 3.9 |
| variability | | | | | | | | |
| Repeatability | 0.295 | 0.288 | 0.259 | 0.260 | 0.255 | 0.261 | 0.270 | 6.4 |

TABLE 10
Intermediate Precision and Operator-to-operator Variability with MBD-biotin Lot 135.

| | | | | | | | | | | | Mean | |
|---|---|---|---|---|---|---|---|---|---|---|---|---|
| Operator | 1 | | | | 2 | | 3 | | | | (μL; | |
| Run | 1 | 2 | 3 | 4 | 1 | 2 | 1 | 2 | 3 | 4 | n = 10) | |
| Volume | 0.200 | 0.194 | 0.204 | 0.260 | 0.193 | 0.196 | 0.270 | 0.265 | 0.164 | 0.175 | 0.212 | Intermediate |
| of MBD- | | | | | | | | | | | | Precision |
| biotin at | | | | | | | | | | | | (% CV) = |
| 50% | | | | | | | | | | | | 18.1 |
| Yield | | | | | | | | | | | | |
| Fraction | | | | | | | | | | | | |
| (μL) | | | | | | | | | | | | |
| Mean | 0.215 | | | | 0.195 | | 0.219 | | | | | Operator- |
| (per | | | | | | | | | | | | to- |
| operator) | | | | | | | | | | | | operator |
| | | | | | | | | | | | | Variability |
| | | | | | | | | | | | | (%) = 6.1 |
| % CV: | 14.3 | | | | 1.1 | | 26.0 | | | | | |
| Intermediate | | | | | | | | | | | | |
| Precision | | | | | | | | | | | | |
| (per | | | | | | | | | | | | |
| operator) | | | | | | | | | | | | |

The hypothesis was that slight changes in MW1 buffer composition, such as final salt concentrations and pH, that may arise from its preparation can impact the ability of the DNA probe's ability to bind MBD. The quality and purity of salts used from different lots of stock were included in this consideration. To test this hypothesis, fresh MW1 and MW3 buffers were prepared as shown in Table 11 below. NO DNA probes were expected to be isolated in the hypomethylated partition (i.e., MW1 buffer), N3 DNA probes were expected to be isolated in the residual partition (a mixture of MW1 and MW3 buffers), and N9 DNA probes were expected to be isolated in the hypermethylated partition (i.e., MW3 buffer). LSE indicates low salt elution buffer and HSE indicates high salt elution buffer.

TABLE 11
Preparation of MW1 and MW3.

| | MW1 | MW3 |
|---|---|---|
| Salt concentration (M) | 0.3 | 2 |
| Total volume (mL) | 4 | 6 |
| LSE (mL) | 3.4 | 0 |
| HSE (mL) | 0.6 | 6 |

The assay was performed as described above in Example 1D-E with a microplate prepared as shown below in Table 12. HSE volumes with 5% more or less than needed were used to simulate pipetting errors. The mixing of these inaccurate HSE volumes with LSE resulted in final MW1 concentrations ranging from 0.287 M to 0.313 M.

TABLE 12
Plate layout.

| | | | | | | | Mean | % |
|---|---|---|---|---|---|---|---|---|
| | 1 | 2 | 3 | 4 | 5 | 6 | (μL) | CV |
| A | LSE 2211676 | LSE 2355370 | LSE 2355370 | LSE 2355370 | LSE 2355370 | LSE 235370 | | |
| B | | HSE; 5% less | HSE: 2.5% less | | HSE: 2.5% more | HSE: 5% more | | |
| C | | | | | | | | |
| D | | | | | | | | |
| E | | | | | | | | |
| F | | | | | | | | |
| 2 | | | | | | | | |
| H | | | | | | | | |
| MW1 Salt | 0.0300 | 0.287 | 0.294 | 0.300 | 0.306 | 0.313 | | |
| Concentration | | | | | | | | |
| (M) | | | | | | | | |
| Volume of | | 0.185 | 0.189 | 0.198 | 0.202 | 0.217 | 0.198 | 6.3 |
| MBD-biotin | | | | | | | | |
| at 50% Yield | | | | | | | | |
| Fraction (μL) | | | | | | | | |

The use of different LSE lots impacted the assay. The volume of MBD-biotin at 50% yield was 0.235 μL and 0.198 μL for LSE lot nos. 2211676 and 2355370, respectively. The calculated mean was 0.217 μL and the % CV was 12.1. The differences between LSE lots contributed to the variability observed in the results.

Changes in MW1 salt concentration also had an impact on the assay. Values for each MW1 concentration were as shown in Table 12 above. As the salt concentration for MW1 increased, the volume of MBD-biotin at 50% yield fraction also increased. A combination of MW1 preparation and source lots of its ingredients appears to explain the variability observed. Using a reference standard in the assay for normalization of yield fraction values would mitigate this variability.

### E. Incubation Temperature

The assay was carried out at several different temperatures as described in Example 1 above. As shown below in Table 13, 50% fraction values were comparable (1.9% CV) across the tested temperatures with a slight increase in % fraction values with increasing temperature. Thus, the assay may be conducted at room temperature, i.e., within the range of 18° C. to 25° C., such as 24° C. There may be a trend of increasing fraction values as temperature increases, so it may be advisable to hold temperature constant to maximize consistency of results. The use of MBD-biotin with differing numbers of freeze-thaw cycles (after storage at −80° C.) may have contributed to the increase in 50% fraction values compared to other runs with single-use proteins (i.e., proteins that did not go through any freeze-thaw cycles). Differences in the number of FT cycles and how the MBD-biotin was handled from operator to operator (e.g., sample mixing method, length of time stored at room temperature) could affect the integrity of the MBD-biotin, which may impact 50% fraction values. For future runs, MBD-biotin with 1× freeze-thaw (after storage at −80° C.) was used and the assay temperature was kept at 24° C.

TABLE 13
MBD-biotin Lot 135 (2X Freeze-thaw)

| | Incubation | | | Mean Volume | |
|---|---|---|---|---|---|
| | Temperature (° C.) | | | of MBD- | |
| | 18 | 24 | 25 | biotin (μL) | % CV |
| 50% Fraction | 0.0674 | 0.0690 | 0.0699 | 0.0688 | 1.9 |
| (Mid-exponential | | | | | |
| Phase) | | | | | |

### Example 5. Use of a Protein Reference Standard for Normalization

MBD-biotin lot135 was used as a reference standard for normalization and intermediate variability was minimized. The volumes of MBD-biotin for MBD-biotin lot 148 were compared to those for MBD-biotin lot 135 (Ratio: test MBD-biotin/reference standard; Table 14 at right column). The ratios were comparable between different operators and different runs.

TABLE 14
Volume of MBD-biotin at 50% Yield Fraction
using Different MBD-biotin Lots.

| | | | | | Ratio of Test |
|---|---|---|---|---|---|
| | | | | | MBD-biotin |
| | | | | | Lot/Reference |
| | | | | | Standard |
| Operator; | Volume of MBD-biotin (μL) | | | | MBD-biotin |
| Run No. | at 50% Yield Fraction | | | | Lot No. 135 |
| Operator 1; Run 3 | 0.164 | 0.176 | | | 1.07 |
| Operator 1; Run 4 | 0.175 | | 0.216 | | 1.23 |
| Operator 2; Run 2 | 0.194 | | 0.226 | | 1.16 |
| Operator 2; Run 3 | 0.204 | 0.207 | | | 1.01 |
| Operator 2; Run 4 | 0.260 | | | 0.248 | 0.95 |
| MBD-biotin Lot | 135 | 148 | 679 | 855 | |

Next, MW1 (0.3 M NaCl) was replaced with LSE (no NaCl) to encourage as much of the N3 DNA probes that are present in a well to bind to MBD and different MBD-biotin lots were tested. As shown in Table 15, the ratios of values measured using MBD-biotin lots 679 and 135 were identical, meaning that MW1 can be replaced by LSE for use in the assay.

TABLE 15
Volume of MBD-biotin at 50% Yield Fraction
using Different MBD-biotin Lots.

| | MBD-biotin Lot | | Ratio of MBD-biotin Lot |
|---|---|---|---|
| Buffer | 135 | 679 | 679/MBD-biotin Lot 135 |
| MW1 | 0.175 | 0.216 | 1.23 |
| LSE | 0.053 | 0.065 | 1.23 |

In additional embodiments, a biotin-conjugated anti-5mC antibody can be used as a reference standard.

### Example 6. Custom Plate Setting for Improved Repeatability

LSE was also evaluated using a custom plate reader setting with LSE Lot 2355370. The custom plate setting involved updating the plate dimensions, such as well diameter, and increasing settle time between fluorescence readings. As shown in Table 16, variability, especially in Blank wells, was reduced.

TABLE 16
% CV with MBD-biotin Lot135

| | | % CV: RFU in triplicates | | | |
|---|---|---|---|---|---|
| | | MW1 | MW1 | LSE | LSE |
| | | with old | with new | with old | with old |
| MBD- | | settings on | settings on | settings on | settings on |
| biotin | | Instru- | Instru- | Instru- | Instru- |
| (μL) | Partition | ment 1 | ment 1 | ment 1 | ment 1 |
| Blank | Bound | 30.2 | 0.5 | 6.7 | 2.2 |
| 0 | | 3.4 | 1.7 | 4.6 | 0.7 |
| 0.05 | | 3.0 | 2.4 | 2.6 | 2.0 |
| 0.1 | | 1.5 | 0.9 | 4.1 | 1.7 |
| 0.2 | | 2.7 | 1.8 | 1.6 | 0.4 |
| 0.4 | | 2.1 | 1.2 | 1.9 | 1.5 |
| 0.8 | | 1.5 | 0.9 | 1.8 | 0.9 |
| 1.6 | | 0.8 | 0.8 | 0.9 | 0.9 |
| Blank | Unbound | 0.7 | 1.6 | 5.7 | 1.4 |
| 0 | | 1.0 | 0.4 | 4.0 | 2.5 |
| 0.05 | | 1.3 | 2.3 | 7.5 | 5.1 |
| 0.1 | | 4.6 | 2.7 | 4.7 | 2.8 |
| 0.2 | | 5.1 | 3.7 | 1.8 | 0.9 |
| 0.4 | | 3.0 | 2.6 | 6.1 | 3.2 |
| 0.8 | | 3.9 | 1.4 | 2.6 | 2.3 |
| 1.6 | | 3.7 | 0.6 | 1.5 | 4.4 |

### Example 7. Qualification of the Plate-Based (Bead-Independent) MBD Assay

This example describes a plate-based MBD assay that may be used with automation for high-throughput processing of samples. The assay was developed based on observations discussed in Examples 4-6, and, similarly, does not require the use of streptavidin beads, a step for magnetic beads separation, or the use of standard curves as no matrix effect was observed. The assay also uses a single plate from start to finish, which is compatible with fluorescence measurements in a plate reader.

The assay was determined to be reliable using the methods described in Example 1A-J above with a few modifications. The first modification was replacing MW1 with LSE. A wash step was also added before addition of MW3 to reduce background of residual unbound N3 DNA probes. Incubation temperature was fixed at 24° C. Then, the 50% (representing mid-exponential phase), 90% (representing transition phase), and 95% (representing stationary phase) fraction points were assessed for reliability. In these experiments, MBD-biotin lots 133, 135, 148, 679, and 855 were tested, in addition to lot nos. Observations from these experiments were as follows.

The R² values of binding curves were calculated using four-parameter (4P) and five-parameter (5P) curve-fitting algorithms to see which algorithm provided a better fit for the standard curve. As shown in Table 17 below and in FIG. 15, the 5P algorithm gave higher R² values and was used for future assay runs.

TABLE 17
R² of Standard Curves.

| | | MBD- | | | |
|---|---|---|---|---|---|
| Oper- | | biotin | Method | | |
| ator | Run No. | Lot No. | Modification | 4P | 5P |
| 1 | 1 | 135 | | 0.9971 | 0.9979 |
| | 1 | 679 | | 0.9980 | 0.9991 |
| | 2 | 135 | LSE lot no. 2211676 | 0.9980 | 0.9991 |
| | 2 | 135 | LSE lot no. 2355370 | 0.9978 | 0.9986 |
| | 3 | 135 | | 0.9926 | 0.9940 |
| | 3 | 855 | | 0.9977 | 0.9989 |
| | 3 | 135 | Without wash step | 0.9957 | 0.9974 |
| | 3 | 135 | With wash step | 0.9984 | 0.9989 |
| | 4 | 135 | | 0.9977 | 0.9990 |
| | 4 | 148 | | 0.9969 | 0.9972 |
| | Robustness | 135 | 18° C. | 0.9932 | 0.9933 |
| | Robustness | 135 | 24° C. | 0.9975 | 0.9991 |
| | Robustness | 135 | 25° C. | 0.9962 | 0.9992 |
| | 5 | 135 | | 0.9987 | 0.9991 |
| | 5 | 133 | 1X freeze-thaw | 0.9982 | 0.9994 |
| | 6 | 135 | 3X freeze-thaw | 0.9964 | 0.9967 |
| | 6 | 135 | | 0.9979 | 0.9985 |
| 2 | 1 | 135 | | 0.9958 | 0.9975 |
| | 1 | 148 | | 0.9955 | 0.9976 |
| | 2 | 135 | | 0.9959 | 0.9974 |
| | 2 | 679 | | 0.9960 | 0.9968 |
| | 3 | 135 | | 0.9984 | 0.9993 |
| | 3 | 855 | | 0.9973 | 0.9991 |
| | 4 | 135 | | 0.9923 | 0.9932 |
| | 4 | 133 | | 0.9914 | 0.9931 |

Using the 5P curve-fitting algorithm, the most reliable fraction point was selected based on three observations. First, run 2 by operator 1 using LSE lot nos. 2211676 and 2355370 and MBD-biotin lot 135 were compared (Table 18). % CVs for 50%, 90%, and 95% fractions were 0.8%. 1.0%, and 2.7%, respectively, which were improvements over a previous run when MW1 was used instead of LSE. In that previous run, the % CV was 12.1%. The 95% fraction was excluded from future considerations due to its high % CV. The remaining data points indicate that different LSE lots do not have an impact on the assay. In future assay runs, LSE was used instead of MW1.

TABLE 18
% CV at 50% and 90% Yield Fractions
for LSE Lot Nos. 2211676 and 2355370.

| LSE Lot No. | 2211676 | 2355370 | Mean | % CV |
|---|---|---|---|---|
| Volume of MBD-biotin at | 0.0478 | 0.0472 | 0.0475 | 0.8 |
| 50% Yield Fraction (μL) | | | | |
| Volume of MBD-biotin at | 0.174 | 0.172 | 0.173 | 1.0 |
| 90% Yield Fraction (μL) | | | | |
| Volume of MBD-biotin at | 0.264 | 0.255 | 0.260 | 2.7 |
| 95% Yield Fraction (μL) | | | | |

Second, MBD-biotin from different lots were used and the volume of MBD-biotin at 50% and 90% yield fractions were compared (Table 19). Ratios of MBD-biotin volumes are calculated by dividing the volume of a test MBD-biotin with the volume of MBD-biotin of lot 135. The ratio of volumes of MBD-biotin were more consistent across operators for the 50% yield fractions than the 90% yield fractions.

TABLE 19
Ratios of MBD-biotin volumes and % CV at 50% and 90% Yield Fractions.

| Operator | 1 | | | | 2 | | | |
|---|---|---|---|---|---|---|---|---|
| MBD- | 679/135 | 855/135 | 148/135 | 133/135 | 679/135 | 855/135 | 148/135 | 133/135 |
| biotin | | | | | | | | |
| Lot | | | | | | | | |
| Ratios | 1.20 | 1.05 | 1.03 | 1.22 | 1.15 | 0.96 | 1.07 | 1.39 |
| at 50% | | | | | | | | |
| Yield | | | | | | | | |
| Fraction | | | | | | | | |
| Ratios | 1.14 | 0.97 | 1.22 | 1.33 | 1.18 | 0.93 | 1.07 | 1.52 |
| at 90% | | | | | | | | |
| Yield | | | | | | | | |
| Fraction | | | | | | | | |

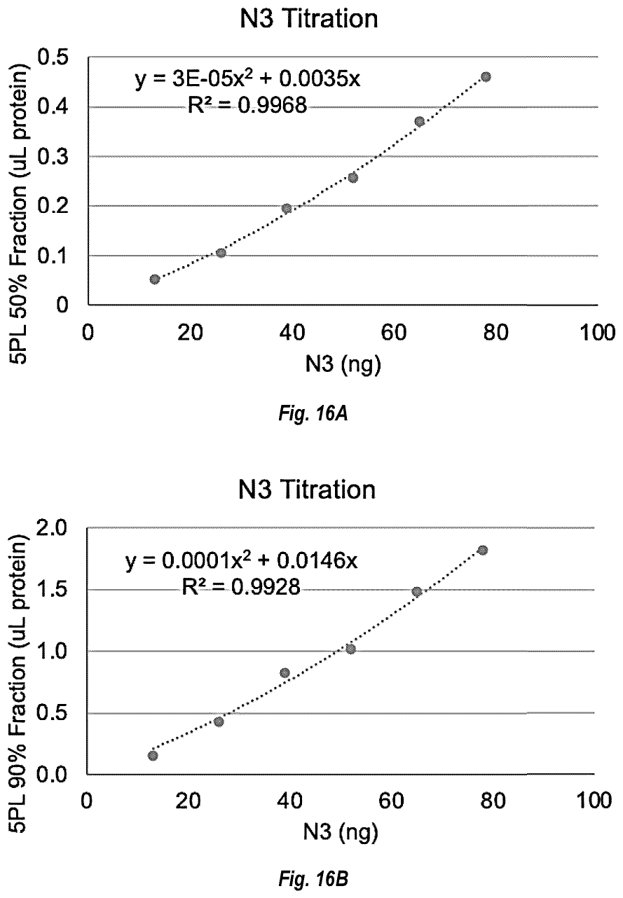

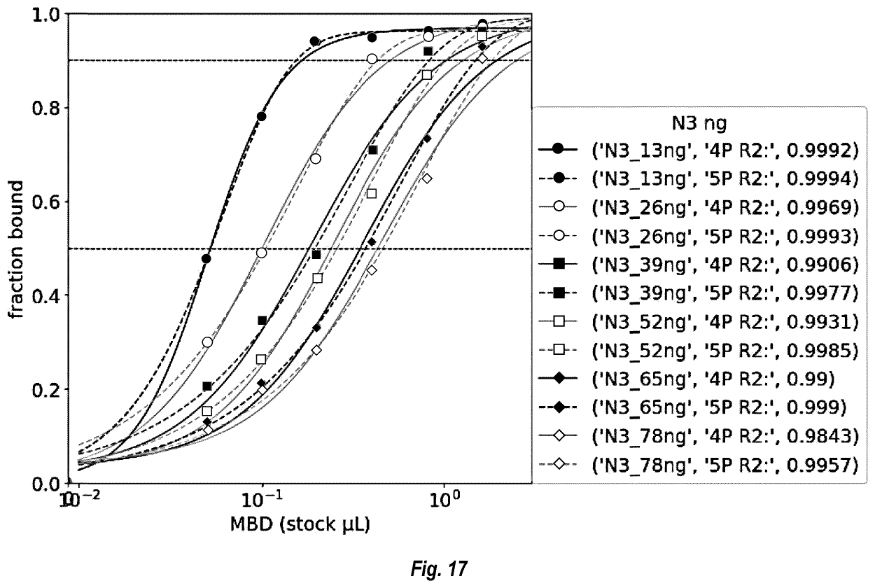

Third, an N3 DNA probe titration experiment was conducted to evaluate if it was possible to define a fraction curve unit as “μL MBD-biotin/ng N3 DNA probe.” This experiment also helped to evaluate whether a 50% fraction point (for mid-exponential phase) or a 90% fraction point (for transition phase) was more reliable. As shown in Table 20 and FIGS. 16A-B, data points for 50% yield fraction provided a curve with an R² of 0.9968 while data points for 90% yield fraction provided a curve with an R² of 0.9928. Thus, the curve for 50% yield fraction was more accurate. We also noted a 2^{nd} order polynomial relationship between the volume of MBD-biotin and the amount of N3 DNA probe. Because that relationship was not linear, the fraction curve unit was not defined as “μL MBD-biotin/ng N3 DNA probe.” This experiment also confirmed that the 5P curve fit was generally preferable to 4P (FIG. 17).

TABLE 20
Volumes of MBD-biotin (μL) from Lot
135 with Titration of N3 DNA Probes.

| N3 (ng), n = 1 | 13 | 26 | 39 | 52 | 65 | 78 |
|---|---|---|---|---|---|---|
| Volume of MBD-Biotin at | 0.05246 | 0.105 | 0.195 | 0.257 | 0.371 | 0.461 |
| 50% Yield Fraction (μL) | | | | | | |
| Volume of MBD-Biotin at | 0.154 | 0.430 | 0.823 | 1.02 | 1.48 | 1.82 |
| 90% Yield Fraction (μL) | | | | | | |

Using the 5P curve-fitting algorithm and data points at 50% yield fraction, the repeatability of the assay was evaluated. Overall, repeatability of all assays without a wash step was less than 17% CV. One run that did not have a wash step (see Table 21 in bold text) had a % CV of 24.5%, which is greater than the maximum threshold of 20%. It is likely that residual unbound N3 carried over to the bound fraction that was later eluted. Further, for the 50% fractions with and without a wash step (see Table 21, operator 1, run 3), the mean volume of MBD-biotin with a wash step was 0.0489 μL, which was higher than the mean volume of MBD-biotin without a wash step (0.0443 μL of MBD-biotin). The mean volume of MBD-biotin calculated from these two runs was 0.0466 μL and their % CV was 6.9. Presumably, residual unbound N3 was removed during the run with the additional wash step and did not contribute to RFU in the bound fraction that was later eluted.

TABLE 21
5 P Curve Fit at 50% Fraction.

| | | | | | | | Mean Volume | |
|---|---|---|---|---|---|---|---|---|
| | | | | | | | of MBD- | |
| | | | | | | | biotin | |
| | | MBD- | | | | | (μL) at | |
| | | biotin | Method | Replicate | | | 50% Yield | % |
| Operator | Run No. | Lot | Modification | 1 | 2 | 3 | Fraction | CV |
| 1 | 1 | 135 | | 0.0563 | 0.0485 | 0.0561 | 0.0536 | 8.3 |
| | 1 | 679 | | 0.0621 | 0.0640 | 0.0670 | 0.0644 | 3.9 |
| | 2 | 135 | LSE lot no. | 0.0481 | 0.0460 | 0.0493 | 0.0478 | 3.5 |
| | | | 2211676 | | | | | |
| | 2 | 135 | LSE lot no. | 0.0466 | 0.0436 | 0.0515 | 0.0472 | 8.5 |
| | | | 2355370 | | | | | |
| | 3 | 135 | | 0.0481 | 0.0298 | 0.0467 | 0.0415 | 24.5 |
| | 3 | 855 | | 0.04416 | 0.04353 | 0.04259 | 0.0434 | 1.8 |
| | 3 | 135 | Without | 0.04752 | 0.04054 | 0.04493 | 0.0443 | 8.0 |
| | | | wash step | | | | | |
| | 3 | 135 | With wash | 0.04746 | 0.04668 | 0.05248 | 0.0489 | 6.4 |
| | | | step | | | | | |
| | 4 | 135 | | 0.0578 | 0.0541 | 0.0546 | 0.0555 | 3.6 |
| | 4 | 148 | | 0.0558 | 0.0636 | 0.0528 | 0.0574 | 9.8 |
| | Robustness | 135 | 18° C. | 0.0653 | 0.0713 | 0.0656 | 0.0674 | 5.0 |
| | Robustness | 135 | 24° C. | 0.0695 | 0.0671 | 0.0704 | 0.0690 | 2.5 |
| | Robustness | 135 | 25° C. | 0.0706 | 0.0685 | 0.0707 | 0.0699 | 1.8 |
| | 5 | 135 | | 0.0473 | 0.0479 | 0.0454 | 0.0469 | 2.7 |
| | 5 | 133 | 1X freeze- | 0.0585 | 0.0576 | 0.0555 | 0.0572 | 2.6 |
| | | | thaw | | | | | |
| | 6 | 135 | 3X freeze- | 0.0510 | 0.0616 | 0.0550 | 0.0558 | 9.6 |
| | | | thaw | | | | | |
| | 6 | 135 | | 0.0558 | 0.0600 | 0.0612 | 0.0590 | 4.8 |
| 2 | 1 | 135 | | 0.0581 | 0.0629 | 0.0693 | 0.0634 | 8.9 |
| | 1 | 148 | | 0.0634 | 0.0659 | 0.0745 | 0.0679 | 8.5 |
| | 2 | 135 | | 0.0697 | 0.0771 | 0.0822 | 0.0764 | 8.2 |
| | 2 | 679 | | 0.0838 | 0.0902 | 0.0895 | 0.0879 | 4.0 |
| | 3 | 135 | | 0.0730 | 0.0749 | 0.0756 | 0.0745 | 1.8 |
| | 3 | 855 | | 0.0695 | 0.0746 | 0.0702 | 0.0714 | 3.9 |
| | 4 | 135 | | 0.0472 | 0.0660 | 0.0577 | 0.0570 | 16.5 |
| | 4 | 133 | | 0.0700 | 0.0746 | 0.0926 | 0.0791 | 15.1 |

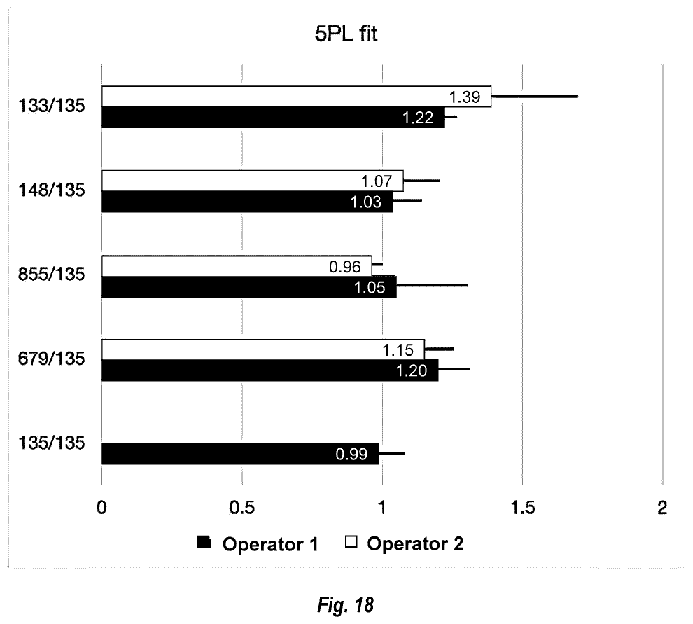

The assay was executed using MBD-biotin lot 135 with an additional wash step (1× freeze-thaw; after storage at −80° C.) and a 5P curve fit. As shown in Table 22, the calculated % CV values at 50% fraction yield for intermediate precision and for operator-to-operator variability were less than 20%. Because MBD-biotin normalization was adopted in the assay (as described above in Example 5), the variability observed across operators did not affect assessment of MBD-biotin lots. Additionally, the ratios of MBD-biotin volumes of different lots at 50% yield fraction were comparable across two different operators (Table 19 above and FIG. 18).

TABLE 22
% CV for Intermediate Precision and Operator-to-operator Variability.

| | Operator | | | | | | | | | |
|---|---|---|---|---|---|---|---|---|---|---|
| | | | | | | | | | Mean | |
| | | | | | | | | | Volume | |
| | 1 (n = 4) | | | | 2 (n = 4) | | | | of MBD- | |
| | Run | | | | | | | | biotin | |
| | 1 | 2 | 3 | 4 | 1 | 2 | 3 | 4 | (μL) | % CV |
| Volume | 0.0489 | 0.0555 | 0.0469 | 0.0556 | 0.0634 | 0.0764 | 0.0745 | 0.0570 | 0.0598 | For |
| of | | | | | | | | | | Intermediate |
| MBD- | | | | | | | | | | Precision = |
| biotin at | | | | | | | | | | 18.2 |
| 50% | | | | | | | | | | |
| Fraction | | | | | | | | | | |
| (μL) | | | | | | | | | | |
| Mean | 0.0517 | | | | 0.0678 | | | | | For |
| Volume | | | | | | | | | | Operator- |
| of | | | | | | | | | | to-operator |
| MBD- | | | | | | | | | | Variability = |
| biotin | | | | | | | | | | 19.0 |
| For Each | | | | | | | | | | |
| Operator | | | | | | | | | | |
| (μL) | | | | | | | | | | |
| % CV: | 8.7 | | | | 13.6 | | | | | |
| Intermediate | | | | | | | | | | |
| Precision | | | | | | | | | | |
| For Each | | | | | | | | | | |
| Operator | | | | | | | | | | |

### Example 8. Refinement of the Plate-Based (Bead-Independent) MBD Assay

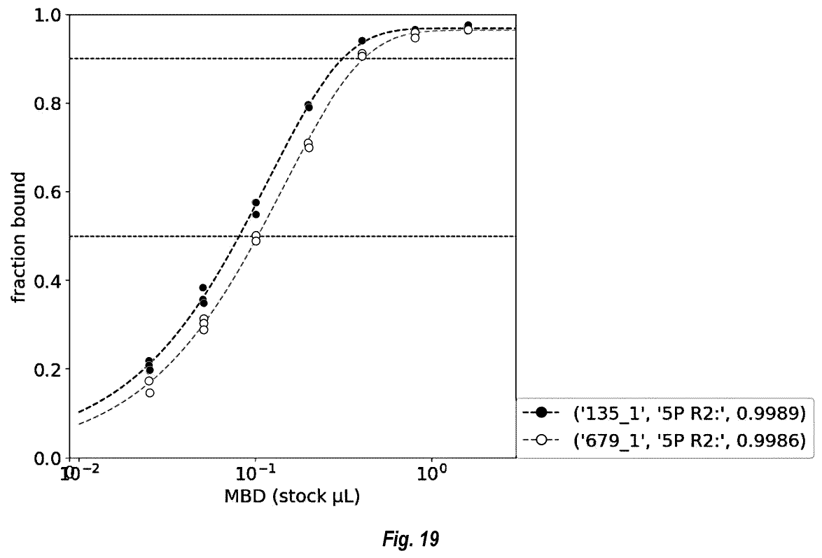

The plate-based MBD assay in Example 7 above was refined with the following considerations. First, the 50% fraction point of the 5P-fitted standard curve was shifted to the center of the curve (FIG. 19). Based on the N3 DNA probe titration experiment discussed in Example 7, the 50% fraction point was centered by increasing the amount of N3 DNA probes to an amount from 13 to 26 ng, which in this case was 20 ng, such that R² was greater than 0.999. Also, 0.05 μL of MBD-biotin was used as the lowest volume on the curve instead of 0 μL, thereby resetting the zero-intercept on the (FIG. 19).

Second, to reflect the true amounts of MBD-biotin used in the assay, the fraction unit for MBD-biotin was defined as “pmol of MBD-biotin” instead of “μL of MBD-biotin.” The binding capacity of streptavidin in the streptavidin-coated plate (Thermo Scientific Cat #15503), according to manufacturer's specifications, was about 125 pmol D-biotin per well. Because the highest amount of MBD-biotin in a well according to the methods disclosed therein was 38 μmol, the amount of MBD-biotin added to each well was completely bound by the streptavidin in the well.

Third, the assay was carried out using different instruments and evaluated for instrument-related variability according. As shown in Table 24, the calculated % CV indicated that the assay was reproducible across different instruments. Further, while variability between instruments was as high as 5.9% CV, the variability was only 1.3% CV when the protein ratio method was used (Table 25). In the preceding examples, binding curves were plotted without centering the 50% yield fraction point. According to those methods, the ratios of MBD-biotin volumes for protein lots 679 and 855 were 1.20 for operator 1 and 1.15 for operator 2 (see Table 20 above). After the curve was shifted to center around the 50% yield fraction point, the revised ratios of 1.26-1.28 should be more accurate.

TABLE 24
% CV with Different Instruments using 20 ng N3 DNA Probes.

| MBD- | | 50% Fraction (pmol) | | | | |
|---|---|---|---|---|---|---|
| biotin | | Replicate | Replicate | Replicate | Mean | % CV: |
| Lot | Instrument | 1 | 2 | 3 | (pmol) | Repeatability |
| 135 | 2 | 2.01 | 1.85 | 1.94 | 1.93 | 4.3 |
| 679 | 2 | 2.40 | 2.55 | 2.49 | 2.48 | 3.0 |
| 135 | 1 | 1.93 | 1.74 | 1.77 | 1.81 | 5.5 |
| 679 | 1 | 2.28 | 2.33 | 2.23 | 2.28 | 2.3 |

TABLE 25
Assay Variability Across Different Instruments.

| | | | Mean | % CV: Instrument-to- |
|---|---|---|---|---|
| MBD-biotin Lot | Post | Pre | (pmol) | instrument Variability |
| 135 | 1.93 | 1.81 | 1.87 | 4.6 |
| 679 | 2.48 | 2.28 | 2.38 | 5.9 |
| Ratio for 135/679 | 1.28 | 1.26 | 1.27 | 1.3 |

### Example 9. Bead-Based MBD Assay with a Triplex Probe Mix

This example describes the bead-based MBD assay with a triple probe mix comprising three different DNA probes: NO-FAM, N3-Cy5, and N9-HEX. The assay was tested with 5 different lots of MBD lots and showed different levels of DNA probe binding efficiency for the MBDs. Methods for this assay are described above in Example 1.L.

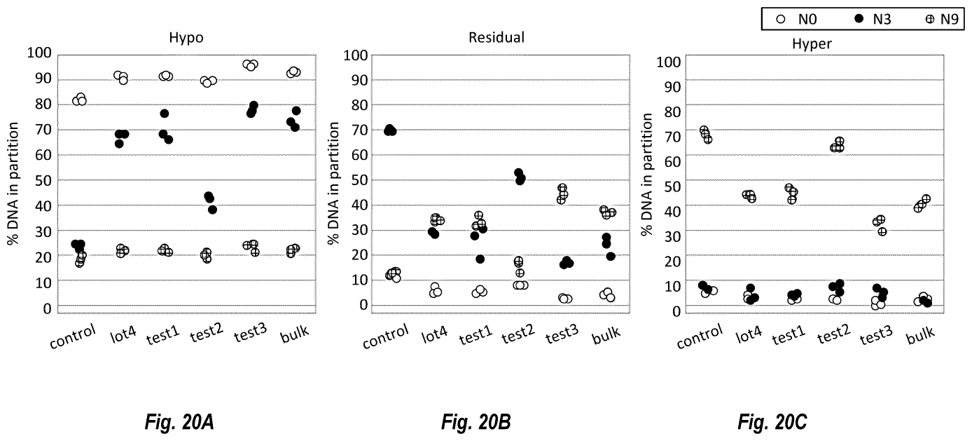

As shown in FIGS. 20A-C, the use of the triplex probe mix allowed for the collection of DNA probes in the expected partitions.

### EQUIVALENTS

The foregoing written specification is considered to be sufficient to enable one skilled in the art to practice the embodiments. The foregoing description and Examples detail certain embodiments and describes the best mode contemplated by the inventors. It will be appreciated, however, that no matter how detailed the foregoing may appear in text, the embodiment may be practiced in many ways and should be construed in accordance with the appended claims and any equivalents thereof.

As used herein, the term about refers to a numeric value, including, for example, whole numbers, fractions, and percentages, whether or not explicitly indicated. The term about generally refers to a range of numerical values (e.g., +/−5-10% of the recited range) that one of ordinary skill in the art would consider equivalent to the recited value (e.g., having the same function or result). When terms such as at least and about precede a list of numerical values or ranges, the terms modify all of the values or ranges provided in the list. In some instances, the term about may include numerical values that are rounded to the nearest significant figure.

1. A method of assaying a methylated DNA-binding protein, comprising:
a. contacting the methylated DNA-binding protein with a sample comprising at least a first oligonucleotide;
b. obtaining at least hypomethylated and hypermethylated partitions of the sample; and
c. quantifying the first oligonucleotide in the hypomethylated and hypermethylated partitions.

2. The method of claim 1, wherein the methylated DNA-binding protein is immobilized on a solid support.

3. The method of claim 2, wherein the solid support comprises plate wells.

4. The method of claim 1, wherein the first oligonucleotide is quantified by measuring absorbance.

5. The method of claim 1, wherein the first oligonucleotide is labeled, optionally wherein the first oligonucleotide is fluorescently or radioactively labeled.

6. (canceled)

7. The method of claim 1, wherein
a. the hypomethylated partition comprises a first salt concentration, and
b. the hypermethylated partition comprises a second salt concentration, wherein the second salt concentration is higher than the first salt concentration.

8. The method of claim 7, wherein
a. the first salt concentration ranges from 0 M to 1 M and/or
b. the second salt concentration ranges from 1.0 M to 3.0 M.

9. (canceled)

10. The method of claim 7, wherein the first salt concentration is about 0.3 M or wherein the second salt concentration is about 2 M.

11. (canceled)

12. The method of claim 7, wherein the first salt or the second salt is a sodium salt, optionally wherein the first salt or the second salt is NaCl.

13. (canceled)

14. The method of claim 1, wherein the first oligonucleotide is methylated.

15. The method of claim 14, wherein the sample further comprises a labeled unmethylated oligonucleotide and the first methylated nucleotide is labeled, further wherein
the labeled unmethylated oligonucleotide is differentially labeled relative to the first methylated oligonucleotide, optionally further comprising quantifying the labeled unmethylated oligonucleotide in the hypomethylated and hypermethylated partitions.

16. (canceled)

17. The method of claim 14, wherein the first methylated oligonucleotide comprises a methylated CpG, optionally wherein the first methylated oligonucleotide comprises at least 2 methylated CpGs or the first methylated oligonucleotide comprises 3 or 9 methylated CpGs.

18. (canceled)

19. (canceled)

20. (canceled)

21. The method of claim 14, wherein the first methylated oligonucleotide is labeled and the sample further comprises a second labeled methylated oligonucleotide, wherein
the second labeled methylated oligonucleotide has a greater number of methylated positions than the first labeled methylated oligonucleotide, further wherein
the second labeled methylated oligonucleotide is differentially labeled relative to the first labeled methylated oligonucleotide, optionally further comprising quantifying the second labeled methylated oligonucleotide in the hypomethylated and hypermethylated partitions.

22. (canceled)

23. The method of claim 19, wherein the sample further comprises a labeled unmethylated oligonucleotide, wherein
the labeled unmethylated oligonucleotide is differentially labeled relative to the first labeled methylated oligonucleotide and the second labeled methylated oligonucleotide, optionally further comprising quantifying the labeled unmethylated oligonucleotide in the hypomethylated and hypermethylated partitions.

24. (canceled)

25. The method of claim 17, wherein each of the first and second labeled methylated oligonucleotides comprises at least one methylated CpG.

26. (canceled)

27. (canceled)

28. The method of claim 17, wherein the first labeled methylated oligonucleotide comprises 3 methylated CpGs or wherein the second labeled methylated oligonucleotide comprises 9 methylated CpGs.

29. (canceled)

30. (canceled)

31. The method of claim 1, further comprising obtaining an intermediate partition or measuring a binding activity of the methylated DNA binding protein.

32. The method of claim 14, wherein the first methylated oligonucleotide and/or a second labeled methylated oligonucleotide binds to a methyl binding domain, a methyl binding protein, and/or an antibody that preferentially binds to 5-methylcytosine (5mC) over unmodified cytosine.

33. The method of claim 1, wherein the methylated DNA binding protein comprises MeCP2, MBD1, MBD2, MBD4, or a methyl-CpG binding zinc finger protein.

34. (canceled)

35. The method of claim 31, wherein the binding activity is determined in units indicating an amount of protein capable of binding a predetermined fraction of a reference methylated oligonucleotide, wherein the reference methylated oligonucleotide comprises at least 1 or at least 2 methylated CpGs or wherein the reference methylated oligonucleotide comprises 3 or 9 methylated CpGs.

36. (canceled)

37. (canceled)

38. (canceled)

39. (canceled)

40. (canceled)

41. (canceled)

42. (canceled)
